(19) United States
(12) Patent Application Publication
(10) Pub. No.: US 2025/0032788 A1
(43) Pub. Date: Jan. 30, 2025

(54) COMPOSITIONS AND METHODS FOR TREATING WITH A COMBINATION OF ALTERNATING ELECTRIC FIELDS AND DNA-DEPENDENT PROTEIN KINASE INHIBITORS

(71) Applicant: NOVOCURE GMBH, Root D4 (CH)

(72) Inventors: Eyal Dor-On, Haifa (IL); Spencer J. Collis, Sheffield (GB); Callum George Jones, Sheffield (GB); Ola Rominiyi, Sheffield (GB); Lilach Avigdor, Haifa (IL)

(21) Appl. No.: 18/759,109

(22) Filed: Jun. 28, 2024

Related U.S. Application Data
(60) Provisional application No. 63/511,363, filed on Jun. 30, 2023.

(51) Int. Cl. A61N 1/36; A61K 31/4985; A61K 31/506; A61K 31/517

(52) U.S. Cl. CPC A61N 1/36002; A61K 31/4985; A61K 31/506; A61K 31/517

(57) ABSTRACT

Disclosed herein is the use of alternating electric fields and a DNA-dependent PK inhibitor to treat cancer. Disclosed herein is the use of alternating electric fields and a DNA-dependent PK inhibitor for inducing cell death. Disclosed herein is the use of alternating electric fields and a DNA-dependent PK inhibitor for inhibiting DNA repair in a cancer cell having double stranded DNA breaks. Disclosed herein is the use of alternating electric fields and a DNA-dependent PK inhibitor for increasing efficacy of radiation therapy.

### CROSS-REFERENCE TO RELATED APPLICATIONS

This application claims the benefit of U.S. Provisional Patent Application No. 63/511,363. filed June 30, 2023, which is incorporated by reference herein in its entirety.

### BACKGROUND

DNA is damaged from many different causes throughout a cell cycle, including a tumor cell cycle. In particular, radiotherapy and chemotherapy are used to induce DNA damage in tumor cells and cause tumor cell death. DNA damage is repaired through a variety of DNA repair pathways, depending on the type of DNA damage. DNA repair pathways consist of the direct repair (DR), base excision repair (BER), nucleotide repair (NER), mismatch repair (MMR), and DNA strand-break repair pathways, among others. DNA-Protein Kinase (DNA-PK) regulates the major pathway (nonhomologous end joining) responsible for repair of DNA double-strand breaks induced by radiation.

When the normal repair process is impaired or altered, DNA damage is not repaired, triggering cell death in tumor cells. DNA repair status in tumor cells is associated with the therapeutic response to the anti-cancer drug, establishing DNA repair pathways as promising targets for cancer treatment.

Tumor treating fields (TTFields), also referred to as alternating electric fields, delay DNA damage repair following radiation treatment of glioma cells. TTFields influence cellular DNA repair capacity by altering the homologous repair pathway.

Thus, altering both homologous and non-homologous repair pathways can be an effective cancer treatment.

### BRIEF SUMMARY

Disclosed are methods of treating a subject having cancer comprising applying an alternating electric field to a target site of the subject for a period of time, wherein the target site comprises one or more cancer cells, and administering a therapeutically effective amount of a DNA-dependent PK inhibitor to the subject.

Disclosed are methods of inducing cell death of a cancer cell comprising exposing the cancer cell to an alternating electric field for a period of time, and exposing the cancer cell to a DNA-dependent PK inhibitor.

Disclosed are methods of inhibiting DNA repair in a cancer cell exposed to or previously exposed to radiation comprising exposing the cancer cell to an alternating electric field for a period of time, and exposing the cancer cell to a DNA-dependent PK inhibitor.

Disclosed are methods of increasing the efficacy of radiation therapy in a subject comprising applying an alternating electric field to a target site of the subject for a period of time, wherein the target site comprises a site receiving or that has received radiation therapy, and administering a therapeutically effective amount of a DNA-dependent PK inhibitor to the subject.

Additional advantages of the disclosed methods and compositions will be set forth in part in the description which follows, and in part will be understood from the description, or may be learned by practice of the disclosed method and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed methods and compositions.

FIG. 1 shows a schematic of DNA repair mechanisms.

FIG. 2 shows a schematic of DNA repair mechanisms.

FIG. 3 shows an example of treating cells with and without radiation and with and without a DNA dependent PK inhibitor. FIG. 3 shows a 3D clonogenic survival of MGMT negative CX18 core 1 and edge 2 primary glioma stem-like cells (GSCs) treated with DNA-PKi or IR alone or in combination (1 hr DNA-PKi prior to IR exposure). The upper panels show western blots of the indicated proteins highlighting effective inhibition of DNA-PK kinase activity (exhibited by reduced IR-induced autophosphorylation at Ser2056) at the indicated doses of DNA-PKi. FIG. 3 also shows a proposed study for using TTFields alongside DNA-PK inhibition and radiation.

FIG. 4 shows an example of treating cells with and without radiation and with and without a DNA dependent PK inhibitor. FIG. 4 shows a 3D clonogenic survival of MGMT positive OX5 core and edge primary glioma stem-like cells (GSCs) treated with DNA-PKi or IR alone or in combination (1 hr DNA-PKi prior to IR exposure). The upper panels show western blots of the indicated proteins highlighting effective inhibition of DNA-PK kinase activity (exhibited by reduced IR-induced autophosphorylation at Ser2056) at the indicated doses of DNA-PKi. FIG 4 also shows a proposed study for using TTFields alongside DNA-PK inhibition and radiation.

FIG. 5 show TTFields alongside VX984 (DNAPKi) enhances glioblastoma cell death by radiosensitisation in 3D Alvetex cultured, primary-derived tumour resection CX18 edge 2, CX18 core 1, OX5 edge and OX5 core. Western blot analysis validated VX984 (250 nM) DNAPK inhibition through depletion of pDNAPK (pS2056) signal in response to radiation (5 Gy) in CX18 edge 2, CX18 core 1, OX5 edge and OX5 core. Clonogenic survival assays of cells pre-treated with VX984 (250 nM, 1 hr) before TTFields incubation (200 kHz, 72 hr) in CX18 edge 2, CX18 core 1, OX5 edge and OX5 core cells. n=3, statistical significance denoted through ANOVA one-way analysis. Representative images of cell death post TTFields treatment in CX18 edge 2), CX18 core 2, OX5 edge and OX5 core cells.

FIG. 6 is a table with a summary of the survival data of TTFields alongside VX984 and radiation. The average intratumoural survival fractions (INTRA); OX5 core vs edge and CX18 core vs edge and the average intertumoural survival fraction; OX5 INTRA vs CX18 INTRA are shown. Bliss indices are calculated to ascertain if cell death is synergistic or addictive using the survival fraction product of DMSO+TTF and Combo−TTF as a ratio against Combo+TTF. Ratios<1.1 are seen as additive cell death whereas>1.1 is synergistic.

FIG. 7 is a diagram showing the DNA damage response mechanisms.

FIG. 8 shows a bioinformatics analysis of response to TTFields.

FIG. 9 shows the structure of Nedisertib and CC-115.

FIG. 10 is a table describing the A549 and H1299 cell lines.

FIG. 11 shows Nedisertib titration.

FIG. 12 shows TTFields enhance the cytotoxic and overall effect of nedisertib in A549 cells. 0.97 V/cm, 72 h, 150 kHz, N=6

FIG. 13 shows TTFields enhance the cytotoxic and overall effect of nedisertib in H1299 cells. 0.97 V/cm, 72 h, 150 kHz, N=5

FIG. 14 shows the concomitant treatment of TTFields and nedisertib promotes cell cycle arrest in A549. 0.97 V/cm, 150 kHz, N=3.

FIG. 15 shows the concomitant treatment of TTFields and nedisertib promotes cell cycle arrest in H1299 cells. 0.97 V/cm, 150 kHz, N=3.

FIG. 16 shows CC-115 titration.

FIG. 17 shows TTFields enhance the cytotoxic and overall effect of CC-115 in A549 cells. 0.97 V/cm, 72 h, 150 kHz, N=6.

FIG. 18 shows TTFields enhance the cytotoxic and overall effect of CC-115 in H1299 cells. 0.97 V/cm, 72 h, 150 kHz, N=4.

FIG. 19 shows the concomitant treatment of TTFields and CC-115 promotes cell cycle arrest in A549 cells. 0.97 V/cm, 150 kHz, N=6.

FIG. 20 shows the concomitant treatment of TTFields and CC-115 promotes cell cycle arrest in H1299 cells. 0.97 V/cm, 150 kHz, N=6.

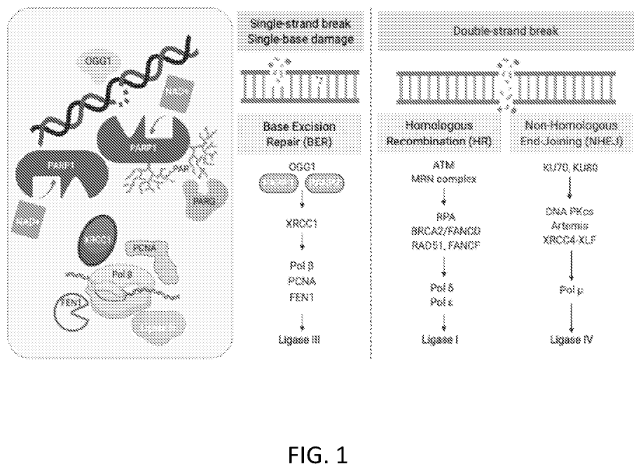

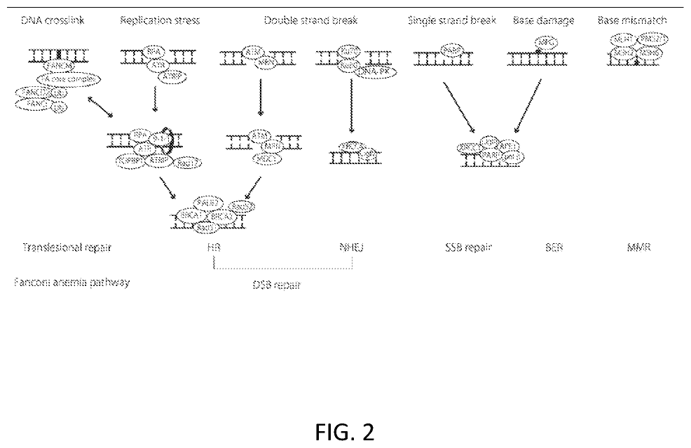

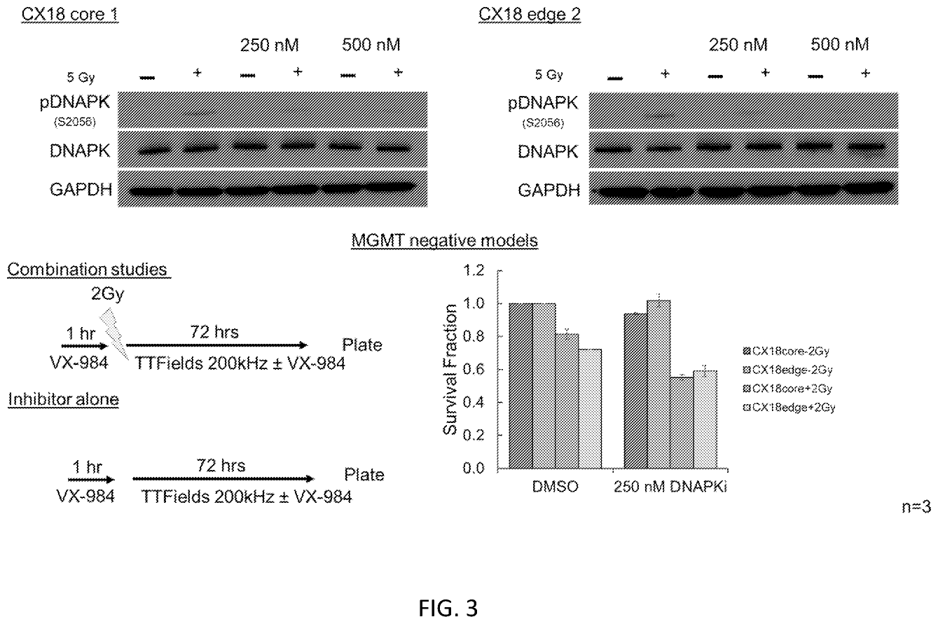

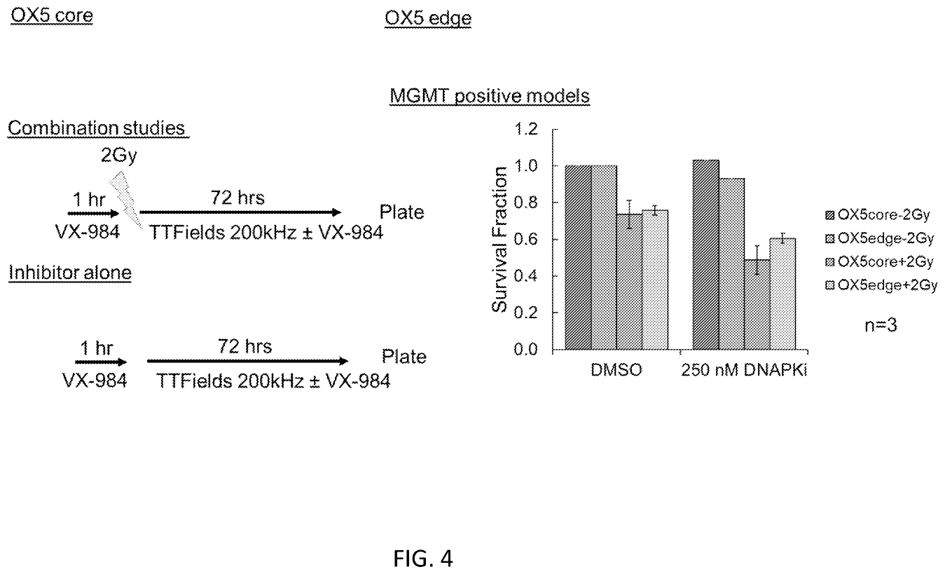

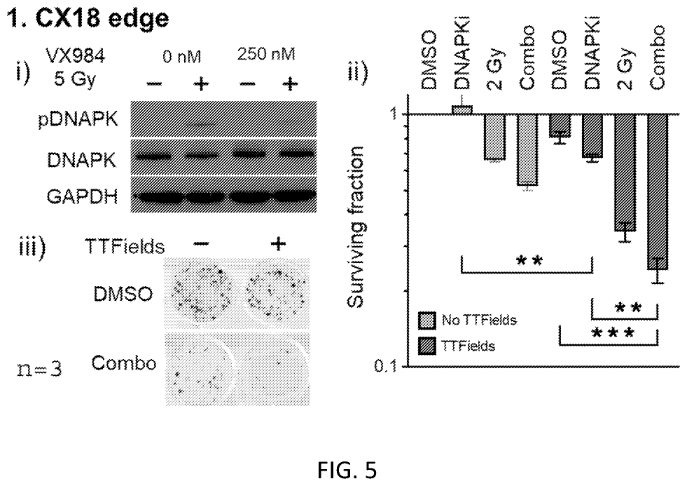

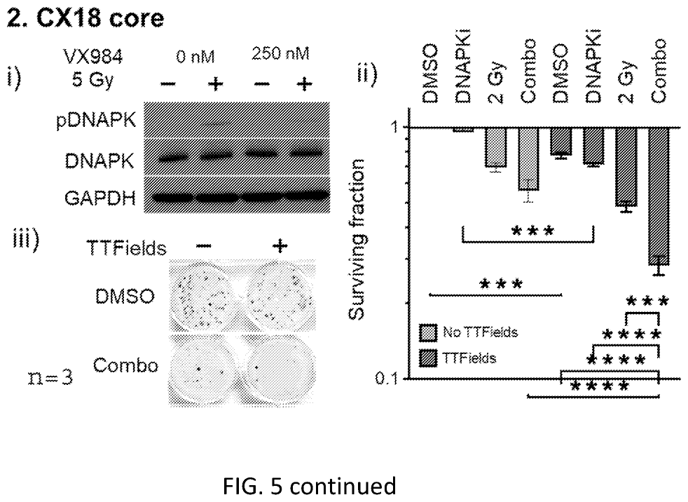

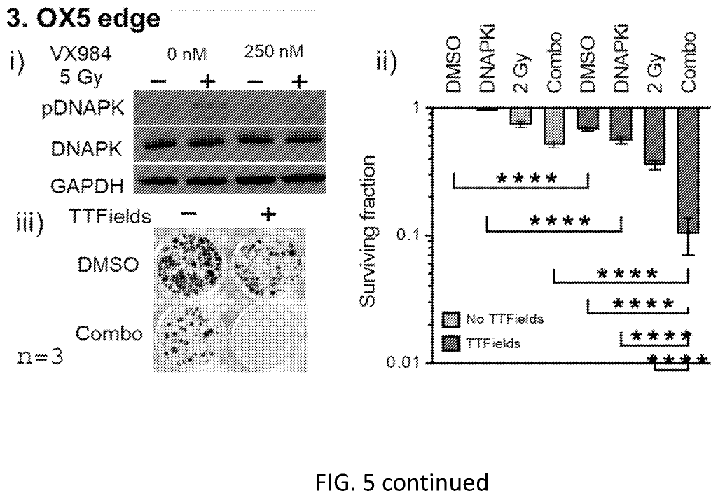

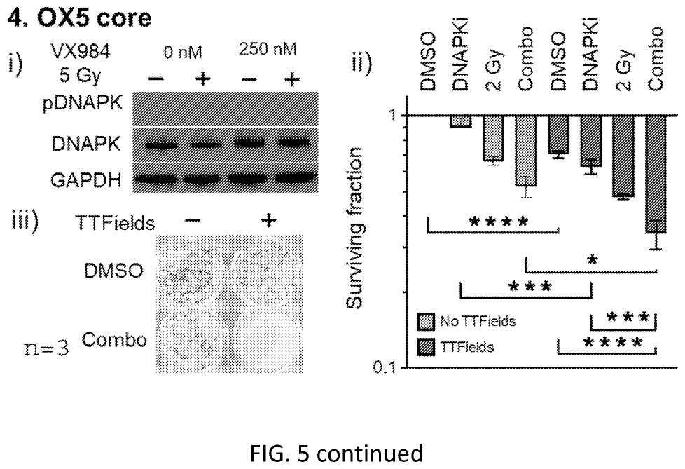

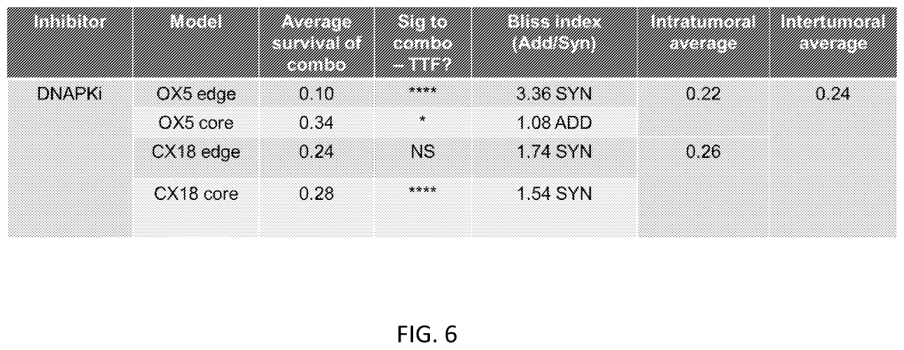

### DETAILED DESCRIPTION

The disclosed methods and compositions may be understood more readily by reference to the following detailed description of particular embodiments and the Example included therein and to the Figures and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a DNA-dependent protein kinase inhibitor” includes a plurality of such inhibitors, reference to “the cell” is a reference to one or more cells and equivalents thereof known to those skilled in the art, and so forth.

The word “or” as used herein means any one member of a particular list and also includes any combination of members of that list.

As used herein, a “target site” is a specific site or location within or present on a subject or patient. For example, a “target site” can refer to, but is not limited to a cell (e.g., a cancer cell), population of cells, organ, tissue, or a tumor. Thus, the phrase “target cell” can be used to refer to target site, wherein the target site is a cell. In some aspects, a “target cell” can be a cancer cell. In some aspects, organs that can be target sites include, but are not limited to, the brain. In some aspects, a cell or population of cells that can be a target site or a target cell include, but are not limited to, a cancer cell (e.g., an ovarian cancer cell). In some aspects, a “target site” can be a tumor target site.

A “tumor target site” is a site or location within or present on a subject or patient that comprises or is adjacent to one or more cancer cells, previously comprised one or more tumor cells, or is suspected of comprising one or more tumor cells. For example, a tumor target site can refer to a site or location within or present on a subject or patient that is prone to metastases. Additionally, a target site or tumor target site can refer to a site or location of a resection of a primary tumor within or present on a subject or patient. Additionally, a target site or tumor target site can refer to a site or location adjacent to a resection of a primary tumor within or present on a subject or patient.

As used herein, an “alternating electric field” or “alternating electric fields” refers to a very-low-intensity, directional, intermediate-frequency alternating electrical fields delivered to a subject, a sample obtained from a subject or to a specific location within a subject or patient (e.g., a target site such as a cell). In some aspects, the alternating electrical field can be in a single direction or multiple directions. In some aspects, alternating electric fields can be delivered through two pairs of transducer arrays that generate perpendicular fields within the target site. For example, for the Optune™ system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

As used herein, an “alternating electric field” applied to a tumor target site can be referred to as a “tumor treating field” or “TTField.” TTFields have been established as an anti-mitotic cancer treatment modality because they interfere with proper micro-tubule assembly during metaphase and eventually destroy the cells during telophase, cytokinesis, or subsequent interphase. TTFields target solid tumors and are described in U.S. Pat. No. 7,565,205, which is incorporated herein by reference in its entirety for its teaching of TTFields.

In-vivo and in-vitro studies show that the efficacy of TTFields therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electric field. For example, the subject of administration can be a subject with ovarian cancer or lung cancer.

By “treat” is meant to administer or apply a therapeutic, such as alternating electric fields and a vector, to a subject, such as a human or other mammal (for example, an animal model), that has cancer or has an increased susceptibility for developing cancer, in order to prevent or delay a worsening of the effects of the disease or infection, or to partially or fully reverse the effects of cancer. For example, treating a subject having glioblastoma can comprise delivering a therapeutic to a cell in the subject.

By “prevent” is meant to minimize or decrease the chance that a subject develops cancer.

As used herein, the terms “administering” and “administration” refer to any method of providing a DNA-dependent PK inhibitor to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat cancer. In further various aspects, a preparation can be administered prophylactically; that is, administered for prevention of cancer. In an aspect, the skilled person can determine an efficacious dose, an efficacious schedule, or an efficacious route of administration so as to treat a subject. In some aspects, administering comprises contacting, exposing or applying. Thus, in some aspects, exposing a target site or subject to alternating electrical fields or applying alternating electrical fields to a target site or subject or contacting alternating electrical fields to a target site or subject means administering alternating electrical fields to the target site or subject. In some aspects, contacting, exposing and applying can be used interchangeably.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise applying an alternating electric field. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 KHz and 1 MHz. The frequency of the alternating electric fields can also be, but is not limited to, between 50 and 500 kHz, between 100 and 500 kHz, between 100-300 kHz, between 25 kHz and 1 MHZ, between 50 and 190 kHz, between 25 and 190 kHz, between 180 and 220 kHz, or between 210 and 400 kHz. In some aspects, the frequency of the alternating electric fields can be electric fields at or about 50 kHz, 100 kHz, 150 kHz, 200 kHz, 250 kHz, 300 kHz, 350 KHz, 400 kHz, 450 KHz, 500 kHz, or any frequency between. In some aspects, the frequency of the alternating electric field is from about 200 kHz to about 400 kHz, from about 250 kHz to about 350 kHz, and may be around 300 KHz.

In some aspects, the field strength of the alternating electric fields can be between 0.5 and 4 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some instances, the electric field in at least a portion of the target site/subject/cancer cells is induced by an applied voltage of at least 50 V RMS, and optionally, the applied voltage is at least 100 V RMS. In some embodiments, an applied voltage of at least 50 V RMS induces an electric field with a field strength of at least 1 V/cm (e.g., at least 5 V/cm) in at least a portion of the target site/subject/cancer cells

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more. In some aspects, the exposure can be consecutive or cumulative. In some aspects, the consecutive exposure may last for at least 6 hours, at least 12 hours, at least 24 hours, at least 36 hours, at least 48 hours, or at least 72 hours or more. In some aspects, the cumulative exposure may last for at least 42 hours, at least 84 hours, at least 168 hours, at least 250 hours, at least 400 hours, at least 500 hours, at least 750 hours, or more. In some aspects, there can be a break in treatment and the alternating electric fields are applied at least 50%, 60%, 70%, 80% of treatment time. For example, in some aspects, cumulative exposure can be for at least 12 hours in a period of 24 hours.

The disclosed methods comprise applying one or more alternating electric fields to a cell or to a subject. In some aspects, the alternating electric field is applied to a target site or tumor target site. When applying alternating electric fields to a cell, this can often refer to applying alternating electric fields to a subject comprising a cell. Thus, applying alternating electric fields to a target site of a subject results in applying alternating electric fields to a cell.

### C. DNA-Dependent Protein Kinase Inhibitors

The methods and kits disclosed herein comprise administering one or more DNA-dependent protein kinase inhibitors. In some aspects, the DNA-dependent protein kinase inhibitor can be, but is not limited to, Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026, NU 7441, or a combination thereof.

### D. Compositions

In some aspects, the DNA-dependent protein kinase inhibitor can be administered with a pharmaceutically acceptable carrier or diluent in any of the disclosed methods.

Accordingly, disclosed are compositions and formulations comprising a DNA-dependent protein kinase inhibitor with a pharmaceutically acceptable carrier or diluent. Suitable DNA-dependent protein kinase inhibitors include, but are not limited to, any of the DNA-dependent protein kinase inhibitors provided herein. For example, disclosed are pharmaceutical compositions, comprising VX984, and a pharmaceutically acceptable carrier or diluent.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl choline (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, PA 1995, which is hereby incorporated by reference for its teaching of pharmaceutically acceptable carriers. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anaesthetics, and the like. In the methods described herein, delivery of the disclosed compositions to cells can be via a variety of mechanisms. The pharmaceutical composition may be administered in a number of ways depending on whether local or systemic treatment is desired, and on the area to be treated.

### 1. Delivery of Compositions

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### E. Methods

Disclosed are methods comprising applying an alternating electric field and administering a DNA-dependent PK inhibitor to a subject or contacting a cell with a DNA-dependent PK inhibitor. In some aspects, alternating electric fields can inhibit the homologous recombination pathway while DNA-dependent PK inhibitors can inhibit the non-homologous end joining repair pathway, therefore resulting in an increase in cell death since the DNA damage is not being repaired.

### 1. Methods of Treating

Disclosed herein is the use of an alternating electric field and a DNA-dependent PK inhibitor to treat cancer.

Disclosed are methods of treating a subject having cancer comprising applying an alternating electric field to a target site of the subject for a period of time, wherein the target site comprises one or more cancer cells, and administering a therapeutically effective amount of a DNA-dependent PK inhibitor to the subject.

In some aspects, the cancer can be, but is not limited to, ovarian cancer, hepatobiliary cancer, prostate cancer, pancreatic cancer, head and neck cancers, glioblastoma, gliosarcoma, leukemia, or non-small cell lung cancer. Thus, in some aspects, the cancer cells are derived from one or more of these cancers. In some aspects, the subject has ovarian cancer, hepatobiliary cancer, prostate cancer, pancreatic cancer, head and neck cancers, glioblastoma, gliosarcoma, leukemia, or non-small cell lung cancer.

In some aspects, applying an alternating electric field occurs 1, 2, 3, 4, 5, 6, or 7 days prior to administering the DNA-dependent PK inhibitor. In some aspects, applying an alternating electric field occurs 1, 2, 3, 4, 5, 6, or 7 days after administering the DNA-dependent PK inhibitor. In some aspects, applying alternating electric fields occurs 1, 2, 3, or 4 weeks prior to administering the DNA-dependent PK inhibitor. In some aspects, applying alternating electric fields occurs 1, 2, 3, or 4 weeks after administering the DNA-dependent PK inhibitor. In some aspects the alternating electric fields and the DNA-dependent PK inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the DNA-dependent PK inhibitor is present in the bloodstream prior to applying the alternating electric field.

In some aspects, the subject having cancer has undergone or is currently undergoing radiation therapy. In some aspects, the disclosed methods comprise a step of administering radiation therapy. In some aspects, radiation therapy can be administered concomitantly with the alternating electric field. In some aspects, radiation therapy can be administered concomitantly with the DNA-dependent PK inhibitor. In some aspects, radiation therapy and an alternating electric field can be administered concomitantly with the DNA-dependent PK inhibitor. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other.

In some aspects, radiation therapy can be administered 1, 2, 3, 4, 5, 6, or 7 days after or prior to applying an alternating electric field. In some aspects, radiation therapy can be administered 1, 2, 3, or 4 weeks after or prior to applying alternating electric fields. In some aspects, the radiation therapy and DNA-dependent PK inhibitor can be administered concomitantly while the alternating electric field is applied days or weeks before or after. In some aspects, the radiation therapy and alternating electric field can be applied concomitantly while the DNA-dependent PK inhibitor is administered days or weeks before or after. In some aspects, the DNA-dependent PK inhibitor and alternating electric field can be applied concomitantly while the radiation therapy is administered days or weeks before or after. In some aspects, the radiation therapy, DNA-dependent PK inhibitor, and alternating electric fields are administered consecutively (without restriction as to the order), at least one day apart. In some aspects, the radiation therapy occurs before applying the alternating electric field. In some aspects, the radiation therapy occurs after applying the alternating electric field.

In some aspects, the disclosed methods further comprise a step of administering chemotherapy to a subject. In some aspects, the chemotherapy can be administered prior to, after, or simultaneously with the alternating electric field. In some aspects, the chemotherapy can be administered prior to, after, or simultaneously with the DNA-dependent PK inhibitor.

In some aspects, a therapeutically effective amount of a DNA-dependent PK inhibitor refers to an amount that is sufficient or effective to prevent or decrease (delay or prevent, inhibit, decrease or reverse) the effects of DNA-dependent PK, including aiding in DNA repair. For example, CC-115, an inhibitor of DNA-dependent PK, can be administered at 5-10 mg twice a day. In some aspects, a therapeutically effective amount of VX-984 is 50-720 mg daily in a 28 day cycle. In some aspects, a therapeutically effective amount of VX-984 is 120. 240, 480, or 720 mg daily. In some aspects, the VX-984 can be administered on Day 2 to Day 4 for up to six 28-day cycles. In some aspects, VX-984 can be at a concentration of 250 nM, 125 nM or 63 nM.

In some aspects, the one or more cancer cells at the target site have one or more DNA strand breaks, for example one or more single stranded DNA breaks or double stranded DNA breaks. In some aspects, at least some of the DNA strand breaks can be from the alternating electric fields. In some aspects, at least some of the DNA strand breaks can be from radiation therapy. In some aspects, at least some of the DNA strand breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the DNA strand breaks can be from other compounds known to directly or indirectly induce DNA damage. Thus, in some aspects, the subject having cancer has undergone or is currently undergoing a cancer treatment that causes DNA strand breaks.

In some aspects, the one or more cancer cells at the target site have double stranded DNA breaks. In some aspects, at least some of the double stranded DNA breaks can be from the alternating electric fields. In some aspects, at least some of the double stranded DNA breaks can be from radiation therapy. In some aspects, at least some of the double stranded DNA breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the double stranded DNA breaks can be from other compounds known to directly or indirectly induce DNA damage. Thus, in some aspects, the subject having cancer has undergone or is currently undergoing a cancer treatment that causes double stranded DNA breaks.

In some aspects, at least one DNA repair mechanism in the one or more cancer cells at the target site is inhibited. In some aspects, the DNA repair mechanism inhibited is homologous recombination repair, non-homologous end joining repair, or both. For example, the alternating electric fields can inhibit the homologous recombination repair pathway and the DNA dependent PK inhibitor can inhibit non-homologous end joining (recombination) repair, thus inhibiting both DNA repair pathways.

In some aspects, one or more cancer cells at the target site undergo cell death. In some aspects, the prevention of DNA repair by the alternating electric fields and DNA dependent PK inhibitor results in cell death.

In some aspects, the DNA-dependent PK inhibitor can be Nedisertib. VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026, NU 7441, or a combination thereof.

In some aspects, the methods can further comprise administering to the subject a therapeutically effective amount of an ATR inhibitor, PARP inhibitor or a combination thereof. In some aspects, the ATR inhibitor can be, but is not limited to, Schisandrin B, Nu6027, Dactolisib, EPT-46464, VE-821, AZ20, Berzosertib, Torin-2, Ceralasertib (AZD6738), Tetrahydropyrazolo [1,5-a]pyrazines, Azabenzimidazoles, Gartisertib, (M4344 or VX-803), Bayl895344 (Elimsuretib), CGK 733, Camonsertib, ATR-IN-4, VE-821, AZ20, ETP-46464, or ATR inhibitor 1. In some aspects, the PARP inhibitor can be, but is not limited to, Olaparib, niraparib, talazoparib, rucaparib, or AZD9574.

In some aspects, the therapeutically effective amount of a DNA-dependent PK inhibitor, ATR inhibitor or a PARP inhibitor can be administered orally, subcutaneously or intravenously. In some aspects, the DNA-dependent PK inhibitor, ATR inhibitor or a PARP inhibitor can be delivered as a composition in any of the delivery mechanisms described herein.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some instances, the electric field in at least a portion of the target site/subject/cancer cells is induced by an applied voltage of at least 50 V RMS, and optionally, the applied voltage is at least 100 V RMS. In some embodiments, an applied voltage of at least 50 V RMS induces an electric field with a field strength of at least 1 V/cm (e.g., at least 5 V/cm) in at least a portion of the target site/subject/cancer cells.

### 2. Methods of Inducing Cell Death

Disclosed herein is the use of alternating electric fields and a DNA-dependent PK inhibitor for inducing cell death.

Disclosed are methods of inducing cell death of a cancer cell comprising exposing the cancer cell to an alternating electric field for a period of time, and exposing the cancer cell to a DNA-dependent PK inhibitor. In some aspects, the methods can be conducted in vitro. In some aspects, the methods can be conducted in vivo.

In some aspects, the cancer cell can be simultaneously exposed to radiation or was previously exposed to radiation.

In some aspects, the cancer cell is in a subject. Thus, in some aspects, the method occurs in vivo. In some aspects, the cancer cell can be in culture. Thus, in some aspects, the method can occur in vitro.

In some aspects, exposing the cancer cell to an alternating electric field comprises administering the alternating electric field to a target site of the subject, wherein the target site comprises one or more cancer cells. In some aspects, the cancer can be, but is not limited to, ovarian cancer, hepatobiliary cancer, prostate cancer, pancreatic cancer, head and neck cancers, glioblastoma, gliosarcoma, leukemia, or non-small cell lung cancer. Thus, in some aspects, the cancer cells are derived from one or more of these cancers.

In some aspects, exposing a cancer cell to an alternating electric field is the same as applying an alternating electric field to a cancer cell. In some aspects, applying an alternating electric field occurs 1, 2, 3, 4, 5, 6, or 7 days prior to administering the DNA-dependent PK inhibitor. In some aspects, applying an alternating electric field occurs 1, 2, 3, 4, 5, 6, or 7 days after administering the DNA-dependent PK inhibitor. In some aspects, applying an alternating electric field occurs 1, 2, 3, or 4 weeks prior to administering the DNA-dependent PK inhibitor. In some aspects, applying an alternating electric field occurs 1, 2, 3, or 4 weeks after administering the DNA-dependent PK inhibitor. In some aspects, the alternating electric fields and the DNA-dependent PK inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the DNA-dependent PK inhibitor is present in the bloodstream prior to applying the alternating electric fields.

In some aspects, the cancer cell is simultaneously exposed to radiation or was previously exposed to radiation. In some aspects, a cancer cell being exposed to radiation is the same as a subject receiving radiation therapy. In some aspects, radiation exposure can be concomitant with exposure to the alternating electric fields. In some aspects, radiation and alternating electric field exposure can be concomitant with the DNA-dependent PK inhibitor. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, radiation exposure can be 1, 2, 3, 4, 5, 6, or 7 days after or prior to applying alternating electric fields. In some aspects, radiation exposure can be 1, 2, 3, or 4 weeks after or prior to applying alternating electric fields. In some aspects, the radiation exposure and administering DNA-dependent PK inhibitor can be concomitant while the alternating electric field is applied days or weeks before or after. In some aspects, the radiation, DNA-dependent PK inhibitor, and alternating electric field are administered consecutively (without restriction as to the order), at least one day apart. In some aspects, the radiation exposure occurs after applying alternating electric fields.

In some aspects, exposing the cancer cell to a DNA-dependent PK inhibitor means the cancer cell to a therapeutically effective amount of DNA-dependent PK inhibitor. In some aspects, a therapeutically effective amount of a DNA-dependent PK inhibitor refers to an amount that is sufficient or effective to prevent or decrease (delay or prevent, inhibit, decrease or reverse) the effects of DNA-dependent PK, including aiding in DNA repair. For example, CC-115, an inhibitor of DNA PK, can be administered at 5-10 mg twice a day. In some aspects, a therapeutically effective amount of VX-984 is 50-720 mg daily in a 28 day cycle. In some aspects, a therapeutically effective amount of VX-984 is 120, 240, 480, or 720 mg daily. In some aspects, VX-984 can be at a concentration of 250 nM, 125 nM or 63 nM.

In some aspects, the one or more cancer cells at the target site have one or more DNA strand breaks, for example, one or more single strand DNA breaks or double stranded DNA breaks. In some aspects, at least some of the DNA strand breaks can be from the alternating electric fields. In some aspects, at least some of the DNA strand breaks can be from radiation therapy. In some aspects, at least some of the DNA strand breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the DNA strand breaks can be from other cancer therapies such as, but not limited to, Poly (ADP-ribose) polymerase (PARP) inhibitors. Thus, in some aspects, the subject having cancer has undergone or is currently undergoing a cancer treatment that causes DNA strand breaks.

In some aspects, the one or more cancer cells at the target site have double stranded DNA breaks. In some aspects, at least some of the double stranded DNA breaks can be from the alternating electric fields. In some aspects, at least some of the double stranded DNA breaks can be from radiation therapy. In some aspects, at least some of the double stranded DNA breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the double stranded DNA breaks can be from other cancer therapies such as, but not limited to, Poly (ADP-ribose) polymerase (PARP) inhibitors. Thus, in some aspects, the subject having cancer has undergone or is currently undergoing a cancer treatment that causes double stranded DNA breaks.

In some aspects, at least one DNA repair mechanism in the one or more cancer cells at the target site is inhibited. In some aspects, the DNA repair mechanism inhibited is homologous recombination repair, non-homologous end joining repair, or both. For example, the alternating electric fields can inhibit the homologous recombination repair pathway and the DNA dependent PK inhibitor can inhibit non-homologous end joining (recombination) repair, thus inhibiting both DNA repair pathways.

In some aspects, one or more cancer cells undergo cell death. In some aspects, the prevention of DNA repair by the alternating electric fields and DNA dependent PK inhibitor enables cell death.

In some aspects, the DNA-dependent PK inhibitor can be Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026, NU 7441, or a combination thereof.

In some aspects, the methods can further comprise exposing the cancer cells to a therapeutically effective amount of an ATR inhibitor, PARP inhibitor or a combination thereof. In some aspects, the ATR inhibitor can be, but is not limited to, Schisandrin B, Nu6027,Dactolisib, EPT-46464, VE-821, AZ20, Berzosertib, Torin-2, Ceralasertib (AZD6738), Tetrahydropyrazolo [1.5-a]pyrazines, Azabenzimidazoles, Gartisertib, (M4344 or VX-803), Bayl895344 (Elimsuretib), CGK 733, Camonsertib, ATR-IN-4, VE-821, AZ20, ETP-46464, or ATR inhibitor 1 In some aspects, the PARP inhibitor can be, but is not limited to, Olaparib, niraparib, talazoparib, rucaparib, or AZD9574.

In some aspects, wherein the cancer cells are in a subject, the therapeutically effective amount of a DNA-dependent PK inhibitor, ATR inhibitor, or a PARP inhibitor can be administered orally, subcutaneously or intravenously. In some aspects, the DNA-dependent PK inhibitor, ATR inhibitor, or a PARP inhibitor can be delivered as a composition in any of the delivery mechanisms described herein.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some instances. the electric field in at least a portion of the target site/subject/cancer cells is induced by an applied voltage of at least 50 V RMS. and optionally. the applied voltage is at least 100 V RMS. In some embodiments. an applied voltage of at least 50 V RMS induces an electric field with a field strength of at least 1 V/cm (e.g., at least 5 V/cm) in at least a portion of the target site/subject/cancer cells.

### 3. Methods of Inhibiting DNA Repair

Disclosed herein is the use of alternating electric fields and a DNA-dependent PK inhibitor for inhibiting DNA repair in a cancer cell having one or more DNA strand breaks, in particular a double stranded DNA break.

Disclosed are methods of inhibiting DNA repair in a cancer cell exposed to or previously exposed to radiation comprising exposing the cancer cell to an alternating electric field for a period of time, and exposing the cancer cell to a DNA-dependent PK inhibitor. In some aspects, the methods can be conducted in vitro. In some aspects, the methods can be conducted in vivo.

In some aspects, disclosed are methods of inhibiting DNA repair in a cancer cell having at least one double stranded DNA break comprising exposing the cancer cell to an alternating electric field for a period of time, and exposing the cancer cell to a DNA-dependent PK inhibitor.

In some aspects, disclosed are methods of inhibiting DNA repair in a cancer cell having at least one DNA strand break comprising exposing the cancer cell to an alternating electric field for a period of time, and exposing the cancer cell to a DNA-dependent PK inhibitor. In some aspects the DNA strand break is a single stranded DNA break. In some aspects the DNA strand break is a double stranded DNA break.

In some aspects, the cancer cell can be simultaneously exposed to radiation or was previously exposed to radiation.

In some aspects, the cancer cell is in a subject. Thus, in some aspects, the method occurs in vivo. In some aspects, the cancer cell can be in culture. Thus, in some aspects, the method occurs in vitro.

In some aspects, exposing the cancer cell to an alternating electric field comprises administering the alternating electric field to a target site of the subject, wherein the target site comprises one or more cancer cells. In some aspects, the cancer can be, but is not limited to, ovarian cancer, hepatobiliary cancer, prostate cancer, pancreatic cancer, head and neck cancers, glioblastoma, gliosarcoma, leukemia, or non-small cell lung cancer. Thus, in some aspects, the cancer cells are derived from one or more of these cancers.

In some aspects, exposing a cancer cell to an alternating electric field is the same as applying an alternating electric field to a cancer cell. In some aspects, applying an alternating electric field occurs 1, 2, 3, 4, 5, 6, or 7 days prior to administering the DNA-dependent PK inhibitor. In some aspects, applying an alternating electric field occurs 1, 2, 3, 4, 5, 6, or 7 days after administering the DNA-dependent PK inhibitor. In some aspects, applying an alternating electric field occurs 1, 2, 3, or 4 weeks prior to administering the DNA-dependent PK inhibitor. In some aspects, applying an alternating electric field occurs 1, 2, 3, or 4 weeks after administering the DNA-dependent PK inhibitor. In some aspects, the alternating electric fields and the DNA-dependent PK inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, a subject can be tested to determine that the DNA-dependent PK inhibitor is present in the bloodstream prior to applying the alternating electric fields.

In some aspects, the cancer cell is simultaneously exposed to radiation or was previously exposed to radiation. In some aspects, a cancer cell being exposed to radiation is the same as a subject receiving radiation therapy. In some aspects, radiation exposure can be concomitant with exposure to the alternating electric fields. In some aspects, radiation and alternating electric field exposure can be concomitant with the DNA-dependent PK inhibitor. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, radiation exposure can be 1, 2, 3, 4, 5, 6, or 7 days after or prior to applying alternating electric fields. In some aspects, radiation exposure can be 1, 2, 3, or 4 weeks after or prior to applying alternating electric fields. In some aspects, the radiation exposure and administering DNA-dependent PK inhibitor can be concomitant while the alternating electric field is applied days or weeks before or after. In some aspects, the radiation, DNA-dependent PK inhibitor, and alternating electric field are administered consecutively (without restriction as to the order), at least one day apart. In some aspects, the radiation exposure occurs after applying alternating electric fields.

In some aspects, exposing the cancer cell to a DNA-dependent PK inhibitor means contacting the cancer cell to a therapeutically effective amount of DNA-dependent PK inhibitor. In some aspects, a therapeutically effective amount of a DNA-dependent PK inhibitor refers to an amount that is sufficient or effective to prevent or decrease (delay or prevent, inhibit, decrease or reverse) the effects of DNA-dependent PK, including aiding in DNA repair. For example, CC-115, an inhibitor of DNA PK, can be administered at 5-10 mg twice a day. In some aspects, a therapeutically effective amount of VX-984 is 50-720 mg daily in a 28 day cycle. In some aspects, a therapeutically effective amount of VX-984 is 120, 240, 480, or 720 mg daily. In some aspects, VX-984 can be at a concentration of 250 nM, 125 nM or 63 nM.

In some aspects, the one or more cancer cells at the target site have one or more DNA strand breaks, for example a single strand DNA break or a double stranded DNA break. In some aspects, at least some of the DNA strand breaks can be from the alternating electric fields. In some aspects, at least some of the DNA strand breaks can be from radiation therapy. In some aspects, at least some of the DNA strand breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the DNA strand breaks can be from other cancer therapies such as, but not limited to, Poly (ADP-ribose) polymerase (PARP) inhibitors. Thus, in some aspects, the subject having cancer has undergone or is currently undergoing a cancer treatment that causes DNA strand breaks.

In some aspects, the one or more cancer cells at the target site have double stranded DNA breaks. In some aspects, at least some of the double stranded DNA breaks can be from the alternating electric fields. In some aspects, at least some of the double stranded DNA breaks can be from radiation therapy. In some aspects, at least some of the double stranded DNA breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the double stranded DNA breaks can be from other cancer therapies such as, but not limited to, Poly (ADP-ribose) polymerase (PARP) inhibitors. Thus, in some aspects, the subject having cancer has undergone or is currently undergoing a cancer treatment that causes double stranded DNA breaks.

In some aspects, at least one DNA repair mechanism in the one or more cancer cells at the target site is inhibited. In some aspects, the DNA repair mechanism inhibited is homologous recombination repair, non-homologous end joining repair, or both. For example, the alternating electric fields can inhibit the homologous recombination repair pathway and the DNA dependent PK inhibitor can inhibit non-homologous end joining (recombination) repair, thus inhibiting both DNA repair pathways.

In some aspects, one or more cancer cells undergo cell death. In some aspects, the prevention of DNA repair by the alternating electric fields and DNA dependent PK inhibitor enables cell death.

In some aspects, the DNA-dependent PK inhibitor can be Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026, NU 7441, or a combination thereof. In some aspects, the methods can further comprise exposing the cancer cells to a therapeutically effective amount of an ATR inhibitor, PARP inhibitor or a combination thereof. In some aspects, the ATR inhibitor can be, but is not limited to, Schisandrin B, Nu6027, Dactolisib, EPT-46464, VE-821, AZ20, Berzosertib, Torin-2, Ceralasertib (AZD6738), Tetrahydropyrazolo [1.5-a]pyrazines, Azabenzimidazoles, Gartisertib. (M4344 or VX-803), Bayl895344 (Elimsuretib), CGK 733, Camonsertib, ATR-IN-4, VE-821, AZ20, ETP-46464, or ATR inhibitor 1. In some aspects, the PARP inhibitor can be, but is not limited to, Olaparib, niraparib, talazoparib, rucaparib, or AZD9574.

In some aspects, wherein the cancer cells are in a subject, the therapeutically effective amount of a DNA-dependent PK inhibitor, ATR inhibitor, or a PARP inhibitor can be administered orally, subcutaneously or intravenously. In some aspects, the DNA-dependent PK inhibitor, ATR inhibitor, or a PARP inhibitor can be delivered as a composition in any of the delivery mechanisms described herein.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5 and 4 V/cm RMS. In some aspects, the alternating electric field has a field strength of 1 V/cm RMS. In some aspects, the alternating electric field has a field strength of any of those described herein.

In some instances, the electric field in at least a portion of the target site/subject/cancer cells is induced by an applied voltage of at least 50 V RMS, and optionally, the applied voltage is at least 100 V RMS. In some embodiments, an applied voltage of at least 50 V RMS induces an electric field with a field strength of at least 1 V/cm (e.g., at least 5 V/cm) in at least a portion of the target site/subject/cancer cells.

### 4. Methods of Increasing Efficacy of Radiation Therapy

Disclosed herein is the use of alternating electric fields and a DNA-dependent PK inhibitor for increasing efficacy of radiation therapy.

Disclosed are methods of increasing the efficacy of radiation therapy in a subject comprising applying an alternating electric field to a target site of the subject for a period of time, wherein the target site comprises a site receiving or that has received radiation therapy, and administering a therapeutically effective amount of a DNA-dependent PK inhibitor to the subject.

In some aspects, the cancer can be, but is not limited to, ovarian cancer, hepatobiliary cancer, prostate cancer, pancreatic cancer, head and neck cancers, glioblastoma, gliosarcoma, leukemia, or non-small cell lung cancer. Thus, in some aspects, the cancer cells are derived from one or more of these cancers. In some aspects, the subject has ovarian cancer, hepatobiliary cancer, prostate cancer, pancreatic cancer, head and neck cancers, glioblastoma, gliosarcoma, leukemia, or non-small cell lung cancer.

In some aspects, applying an alternating electric field occurs 1, 2, 3, 4, 5, 6, or 7 days prior to administering the DNA-dependent PK inhibitor. In some aspects, applying an alternating electric field occurs 1, 2, 3, 4, 5, 6, or 7 days after administering the DNA-dependent PK inhibitor. In some aspects, applying alternating electric fields occurs 1, 2, 3, or 4 weeks prior to administering the DNA-dependent PK inhibitor. In some aspects, applying alternating electric fields occurs 1, 2, 3, or 4 weeks after administering the DNA-dependent PK inhibitor. In some aspects, the alternating electric fields and the DNA-dependent PK inhibitor are administered concomitantly. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects. a subject can be tested to determine that the DNA-dependent PK inhibitor is present in the bloodstream prior to applying the alternating electric fields.

In some aspects, the subject having cancer has undergone or is currently undergoing radiation therapy. In some aspects, radiation therapy can be administered concomitantly with the alternating electric fields. In some aspects, radiation therapy and alternating electric fields can be administered concomitantly with the DNA-dependent PK inhibitor. In some aspects, concomitantly refers to within 1, 2, 3, 4, 5, 6, 7, 8, 9, 10, 11, 12, 13, 14, 15, 16, 17, 18, 19, 20, 21, 22, 23, or 24 hours of each other. In some aspects, radiation therapy can be administered 1, 2, 3, 4, 5, 6, or 7 days after or prior to applying alternating electric fields. In some aspects, radiation therapy can be administered 1, 2, 3, or 4 weeks after or prior to applying alternating electric fields. In some aspects, the radiation therapy and DNA-dependent PK inhibitor can be administered concomitantly while the alternating electric field is applied days or weeks before or after. In some aspects, the radiation therapy, DNA-dependent PK inhibitor, and alternating electric fields are administered consecutively (without restriction as to the order), at least one day apart. In some aspects, the radiation therapy occurs after applying alternating electric fields.

In some aspects, a therapeutically effective amount of a DNA-dependent PK inhibitor refers to an amount that is sufficient or effective to prevent or decrease (delay or prevent, inhibit, decrease or reverse) the effects of DNA-dependent PK, including aiding in DNA repair. For example, CC-115, an inhibitor of DNA PK, can be administered at 5-10 mg twice a day. In some aspects, a therapeutically effective amount of VX-984 is 50-720 mg daily in a 28 day cycle. In some aspects, a therapeutically effective amount of VX-984 is 120, 240, 480, or 720 mg daily. In some aspects, the VX-984 can be administered on Day 2 to Day 4 for up to six 28-day cycles. In some aspects, VX-984 can be at a concentration of 250 nM, 125 nM or 63 nM.

In some aspects, the one or more cancer cells at the target site have one or more DNA breaks, for example single stranded DNA breaks or double stranded DNA breaks. In some aspects, at least some of the DNA strand breaks can be from the alternating electric fields. In some aspects, at least some of the DNA strand breaks can be from radiation therapy. In some aspects, at least some of the DNA strand breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the DNA strand breaks can be from other cancer therapies such as, but not limited to, Poly (ADP-ribose) polymerase (PARP) inhibitors. Thus, in some aspects, the subject having cancer has undergone or is currently undergoing a cancer treatment that causes DNA strand breaks.

In some aspects, the one or more cancer cells at the target site have double stranded DNA breaks. In some aspects, at least some of the double stranded DNA breaks can be from the alternating electric fields. In some aspects, at least some of the double stranded DNA breaks can be from radiation therapy. In some aspects, at least some of the double stranded DNA breaks can be from a chemotherapy, such as but not limited to, DNA-alkylating agents and DNA-cross linking agents. Thus, in some aspects, the subject having cancer has undergone or is currently undergoing chemotherapy. In some aspects, at least some of the double stranded DNA breaks can be from other cancer therapies such as, but not limited to, Poly (ADP-ribose) polymerase (PARP) inhibitors. Thus, in some aspects, the subject having cancer has undergone or is currently undergoing a cancer treatment that causes double stranded DNA breaks.

In some aspects, at least one DNA repair mechanism in the one or more cancer cells at the target site is inhibited. In some aspects, the DNA repair mechanism inhibited is homologous recombination repair, non-homologous end joining repair, or both. For example, the alternating electric fields can inhibit the homologous recombination repair pathway and the DNA dependent PK inhibitor can inhibit non-homologous end joining (recombination) repair, thus inhibiting both DNA repair pathways. In some aspects, the alternating electric fields can inhibit the homologous recombination repair pathway and the DNA dependent PK inhibitor can inhibit stress-induced DNA breaks.

In some aspects, one or more cancer cells at the target site undergo cell death. In some aspects, the prevention of DNA repair by the alternating electric fields and DNA dependent PK inhibitor enables cell death.

In some aspects, the DNA-dependent PK inhibitor can be Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026, NU 7441, or a combination thereof.

In some aspects, the methods can further comprise administering to the subject a therapeutically effective amount of an ATR inhibitor, PARP inhibitor or a combination thereof. In some aspects, the ATR inhibitor can be, but is not limited to, Schisandrin B, Nu6027,Dactolisib, EPT-46464, VE-821, AZ20, Berzosertib, Torin-2, Ceralasertib (AZD6738), Tetrahydropyrazolo [1.5-a]pyrazines, Azabenzimidazoles, Gartisertib, (M4344 or VX-803), Bayl895344 (Elimsuretib), CGK 733, Camonsertib, ATR-IN-4, VE-821, AZ20, ETP-46464, or ATR inhibitor 1. In some aspects, the PARP inhibitor can be, but is not limited to, Olaparib, niraparib, talazoparib, rucaparib, or AZD9574.

In some aspects, the therapeutically effective amount of a DNA-dependent PK inhibitor, ATR inhibitor, or a PARP inhibitor can be administered orally, subcutaneously or intravenously. In some aspects, the DNA-dependent PK inhibitor, ATR inhibitor, or a PARP inhibitor can be delivered as a composition in any of the delivery mechanisms described herein.

In some aspects, the alternating electric field can have a frequency and field strength. In some aspects, the frequency of the alternating electric field is between 50 and 1 MHz. In some aspects, the frequency of the alternating electric field is 100-1 MHz. In some aspects, the frequency of the alternating electric field is 100-500 kHz. In some aspects, the frequency of the alternating electric field is 200 kHz. In some aspects, the alternating electric field can be any of the ranges described herein.

In some aspects, the alternating electric field has a field strength of between 0.1 and 10 V/cm RMS. In some aspects, the alternating electric field has a field strength of between 0.5and 4 V/cm RMS. In some aspects, the alternating electric field has a field strength of 1 V/cm RMS. In some aspects, the alternating electric field has a field strength of any of those described herein.

In some instances, the electric field in at least a portion of the target site/subject/cancer cells is induced by an applied voltage of at least 50 V RMS, and optionally, the applied voltage is at least 100 V RMS. In some embodiments, an applied voltage of at least 50 V RMS induces an electric field with a field strength of at least 1 V/cm (e.g., at least 5 V/cm) in at least a portion of the target site/subject/cancer cells.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more of DNA-dependent PK inhibitors and one or more materials for delivering alternating electric fields. The materials may include transducer arrays that generate fields within the target site. The materials may include one or more electrodes that are configured to be attached to a subject or to a target site (possibly via an adhesive), the electrodes being coupled to a generator for generating voltage signals that, when used, induce the alternating electric fields for the methods disclosed. In one example, the one or more materials for delivering alternating electric fields is the Optune system. For example disclosed are kits comprising one or more of Nedisertib, VX-984, CC-115, CC-122, AZD7648, matinib, vemurafenib, and gefitinib and one or more materials for delivering alternating electric fields, such as the Optune system.

### EXAMPLES

### A. Example 1

DNA-PK regulates the major pathway (non-homologous end joining) responsible for repair of DNA double-strand breaks induced by radiation. Mutation or inhibition of DNA-PK results in a marked radiosensitization of cells, tumors, and tissues, as demonstrated by the 2 to 3-fold increase in radiosensitivity of cells and tissues of the severe combined immunodeficiency mouse, which is mutated in DNA-PK [Biedermann et al. PNAS 88(4):1394-1397 February 1991].

DNA-PK inhibition inhibits non-homologous end joining (NHEJ) and increases DSBs (DNA double-strand breaks).

TTFields delay DNA damage repair and have been previously shown to provide a synergistic effect with PARP inhibitors and ATR/ATM inhibitors [Biedermann, 1991; Giladi et al. Radiat Oncol. 2017 Dec. 29;12:206; Mumblat et al. Lung Cancer. 2021 Oct;160:99-110]. TTFields induce formation of DNA double strand breaks due to blockage of DNA damage repair mechanisms. TTFields impair DNA damage repair in MPM cells, specifically the FA-BRCA pathway.

Experiments using concomitant treatment of TTFields with the DNA-dependent protein kinase (PK) inhibitor, VX-984 on the doses 250 nM, 125 nM and 63 nM which will be 1X, 0.5X, 0.25X the dose validated by western blot will be performed. There will also be western blot data in glioblastoma cells derived from patients.

Experiments with CC-115 and Nedisertib will be performed and cytotoxicity will be examined by cell count in the flow cytometer, apoptosis assay and colonogenic assays.

TTFields show efficacy with ATR/ATM/PARP inhibition (Karanam et al. Transl Res. 2020 Mar;217:33-46; Karanam et al. Cell Death Dis. 2017 Mar. 30;8(3):e2711; Karanam et al. International J. of Radiation Oncology Biology Physics 2454, volume 111, issue 3,supplement E230-E231). TTFields plus PARP inhibitor data shows synergy (Martinez-Conde et al. International J Radiation Biology Physics. vol 114, issue 3, supplement, Nov. 2022, page e276; U.S. Pat. No. 10,953,209)

A clear increase in phosphorylation of DNA-PK (pS2056) at 1 h and 4 h post radiation therapy (RT) was observed after RT with a similar increase observed when TTFields were applied after RT. (No increase in DNA-PK phosphorylation was observed with TTFields alone) (Giladi et al. Radiat Oncol. 2017 Dec. 29;12:206).

Nedisertib (M3814, Peposertib, MSC2490484A) is currently recruiting patients for phase I clinical trials in leukemia, solid tumors (Ovarian cancer, hepatobiliary cancer, prostate cancer, pancreatic cancer, head and neck cancers), gliosarcoma and glioblastoma. VX-984(M9831) has been tested in advanced solid tumors (phase I, 2017 one study). CC-115, a small-molecule mTOR and DNA-PK inhibitor, is undergoing phase 1 clinical trial which includes 44 advanced solid or hematologic malignancy patients receiving CC-115 monotherapy. Each of these DNA-PK inhibitors can be used in combination with TTFields.

### B. Example 2

Cellular DNA is exposed to various kinds of DNA damage inducers. When DNA damage is recognized, a complex network of signaling cascade is activated and the cell cycle is temporarily arrested to ensure enough time for cells to repair the damage. However, when the damage is too extensive it can eventually lead to cell death and indeed some anti-cancer drugs are used in the clinic to induce DNA damage in cells for the purpose of triggering cell death.

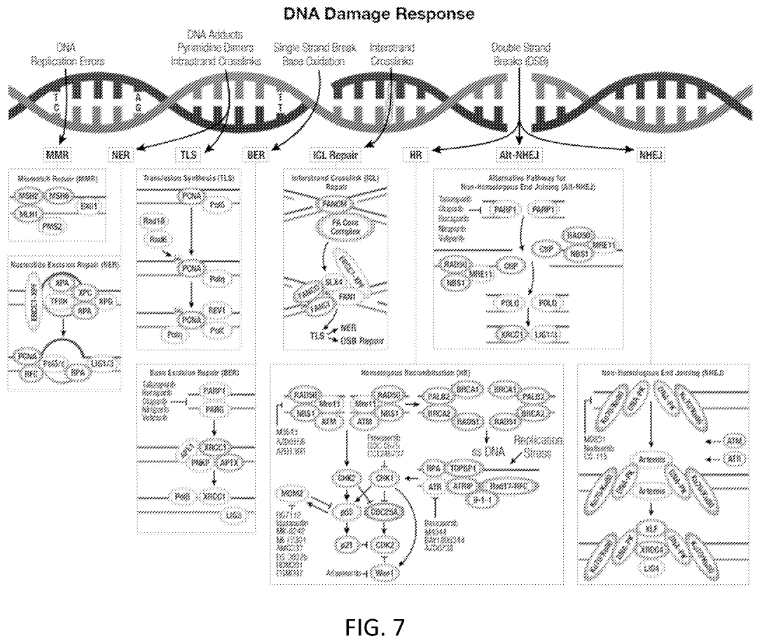

As a defense mechanism, cells have various DNA damage response systems, each is specific for a type of damage and main proteins in the repair pathways are targeted as promising anti cancer treatments (FIG. 7). In this project we are focusing on the repair of double strand breaks by two major systems—the Homologous recombination (HR) and non homologous end joining. It is worth mentioning that during the repair of interstrand crosslinks by the Fanconi anemia proteins, double strand breaks are also formed and repaired afterward by those two systems.

The first line of repair of double strand breaks is the non-homologous end joining (NHEJ) system which ligate the broken ends of the DNA without the need for a homologous template. That is why NHEJ can execute its function throughout the cell cycle, but it is most important during the G1 phase. NHEJ repair is mediated through Ku70/Ku80 heterodimer which recognizes and binds the double strand breaks and recruits DNA-PK proteins at the first stage.

HR on the other hand performs under the guidance of an intact homologous template DNA so it is most active in the S/G2 phase and nearly absent in the G1 phase. This highly regulated pathway is known as an error-free repair pathway and its major players are ATM, RAD51 and BRCA1/2 genes.

TTFields have been shown to down regulate the FANC/BRCA protein levels in cells, thus impairing the repair process by HR and leading to replication stress and DNA damage.

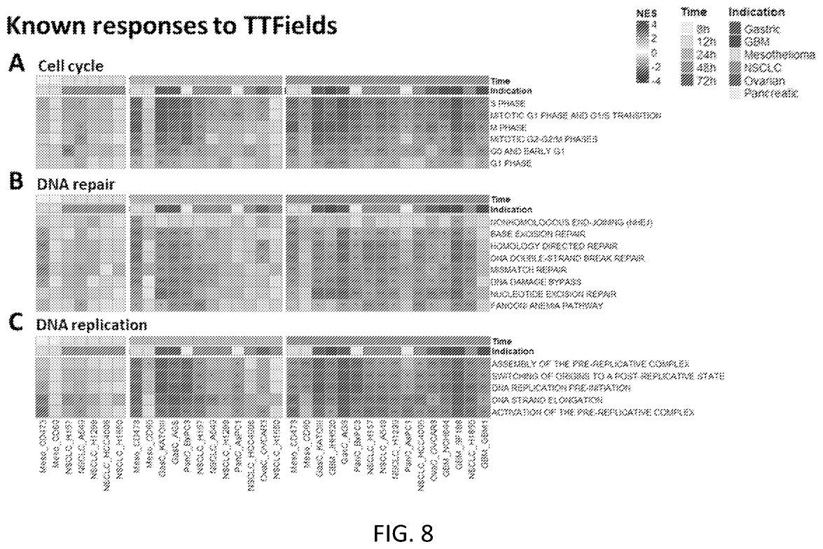

Previous studies have established that TTFields influence cellular DNA repair capacity by altering the HR repair pathway and it has been shown that TTFields don't inhibit DNA-PK phosphorylation and activation in glioma cells. In addition, bioinformatics analysis (FIG. 8) showed that in almost all of the examined cell lines there isn't a significant increase or decrease in the RNA levels of the NHEJ pathway following TTFields, while other DNA damage repair pathways are significantly downregulated.

The rationale for this project is to combine TTFields which impair the HR pathway with DNA-PK inhibitors which impair the NHEJ pathway and thus lead to cell death.

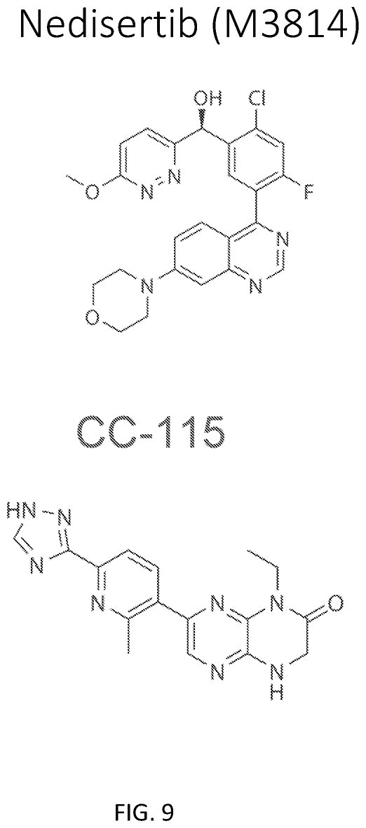

Nedisertib and CC-115 were the inhibitors used in these studies (FIG. 9). Nedisertib is a potent inhibitor of the catalytic unit of DNA-PK. There are several phase I/II clinical trials that are currently recruiting patients to test this inhibitor in leukemia and solid tumors of different types.

CC-115 is a dual inhibitor of DNA-PK and mTOR. A phase I clinical trial which investigated the safety and preliminary efficacy of CC-115 was completed.

This inhibitor was tested because of its dual inhibition with mTOR which regulates proliferation and survival and is related to the AKT signaling pathway that has been established to be affected by TTFields.

The study objectives are to evaluate the cytotoxicity, apoptotic and clonogenic effect of the concomitant treatment of TTFields and Nedisertib or CC-115 in NSCLC (A549 and H1299 cells). In addition, this study investigates the mechanism of action of the concomitant treatment.

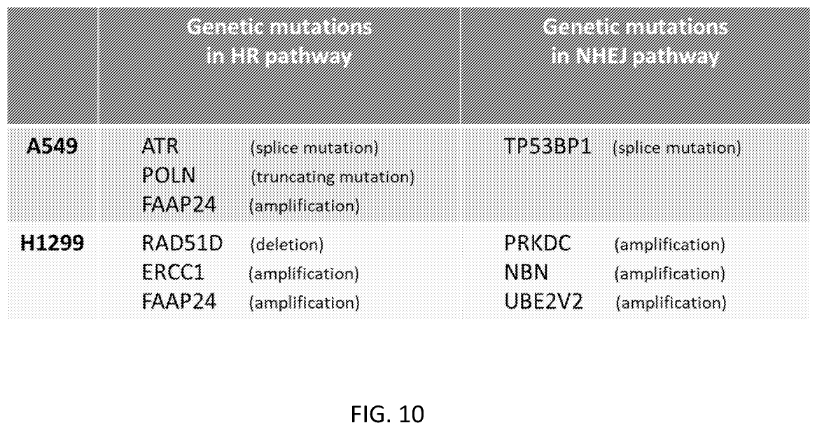

In this project A549 and H1299 cell lines were used which were maintained in F12K or RPMI medium supplemented with 10% FCS. respectively (FIG. 10). The genetic mutations in the HR and NHEJ pathways on the cells are shown in the table. Both cell lines have mutations which can impair the HR pathway and in H1299 cells there is an amplification mutation in PRKDC which is the catalytic unit of DNA-PK.

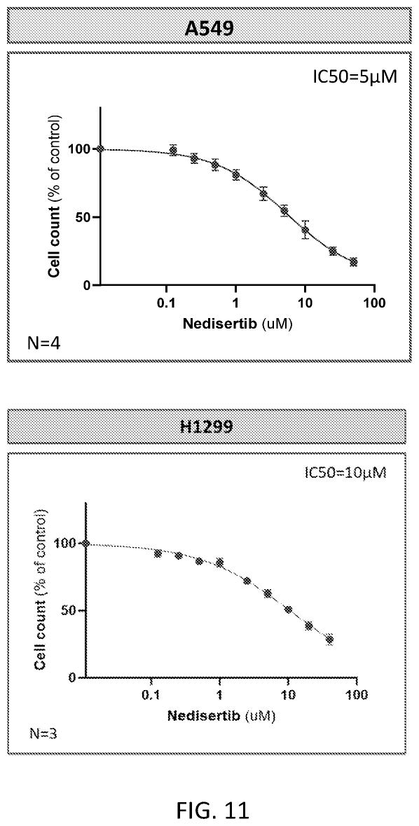

This work was began with the titration of nedisertib. For these experiments 15,000 of A549 or H1299 cells/dish were seeded in 12-well plate and incubated overnight to allow cell attachment. The cells were then treated with various concentrations of nedisertib for 72 hr. The remaining cells were counted by flow cytometer (Guava easyCyte HT) (FIG. 11). The IC50 for both cell lines was similar to previously reported results and the IC50 for H1299 is higher than that of A549, probably due to the amplification mutation in the catalytic unit of DNA-PK.

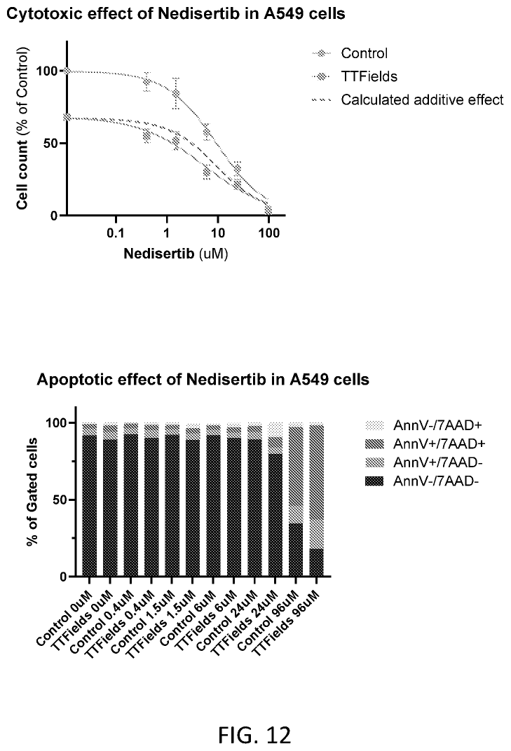

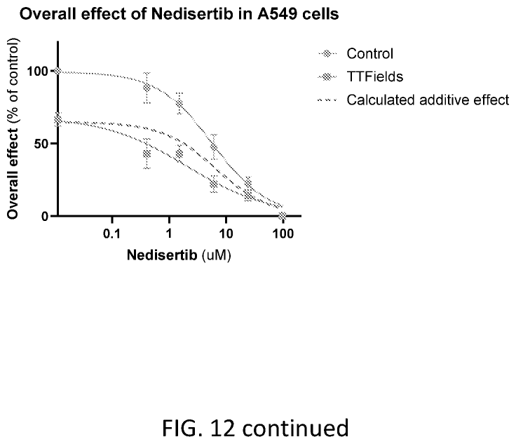

The cytotoxic, apoptotic and clonogenic effect of the concomitant treatment of nedisertib and TTFields were investigated and the overall effect was calculated (FIG. 12).

In these experiments 15,000 of A549 cells/dish were seeded in inovitro dishes and incubated overnight to allow cell attachment. The cells were then treated with TTFields (0.97V/cm RMS, 150 kHz, 72 h), alone or with the addition of different concentrations of nedisertib, Efficacy was measured by analyzing cell count by flow cytometer (Guava easy Cyte HT), colony formation, and apoptosis induction by annexin V/7AAD staining. The overall effect was calculated by multiplying cell count with colony formation. The results showed that TTFields synergistically enhanced the cytotoxic and overall effect of nedisertib.

The apoptotic effect of the concomitant treatment is only significant in the higher doses of nedirsetib, implying that the treatment mainly affects the proliferation of the cells.

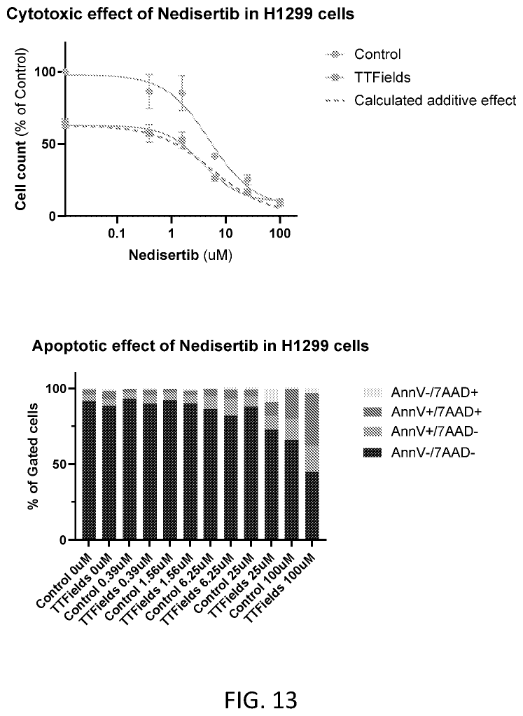

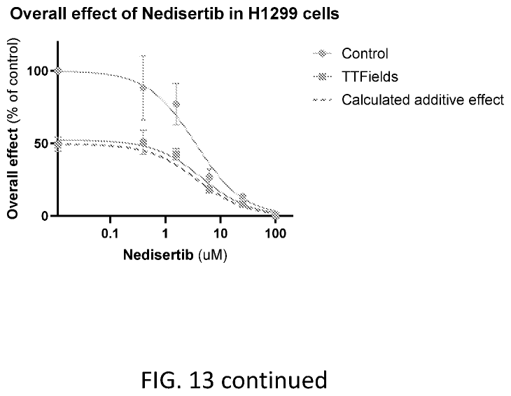

As for H1299, we can see an additive effect for the concomitant treatment of TTFields and nedisertib (FIG. 13). Similarly to A549, the apoptotic effect of the concomitant treatment is only significant in the higher doses of nedirsetib.

In these experiments 15,000 of H1299 cells/dish were seeded in inovitro dishes and incubated overnight to allow cell attachment. The cells were then treated with TTFields (0.97 V/cm RMS, 150 KHz, 72 h), alone or with the addition of different concentrations of nedisertib. Efficacy was measured by analyzing cell count by flow cytometer (Guava easy Cyte HT), colony formation, and apoptosis induction by annexin V/7AAD staining. The overall effect was calculated by multiplying cell count with colony formation.

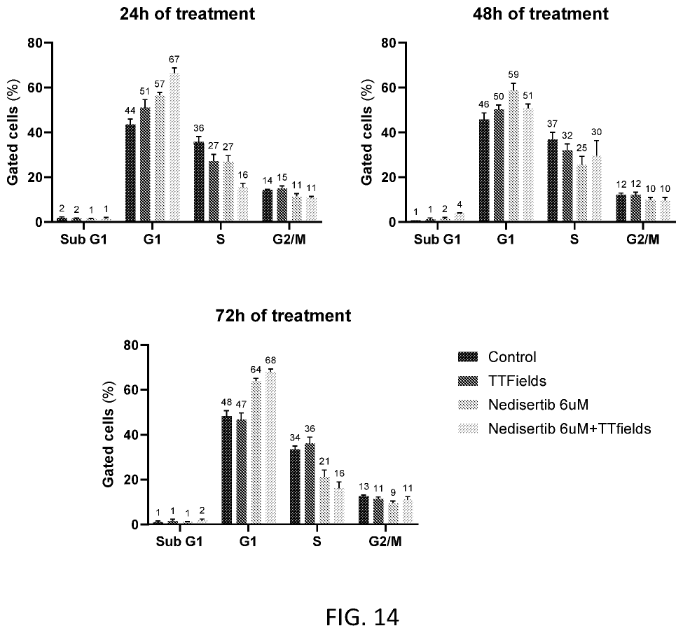

The mechanism of action of the concomitant treatment was investigated and cell cycle analysis was performed. In these experiments 15,000 of A549 cells/dish were seeded in inovitro dishes and incubated overnight to allow cell attachment. The cells were then treated with TTFields (0.97 V/cm RMS. 150 kHz) for different times, alone or with the addition of 6 μM of nedisertib (FIG. 14). Cell cycle distribution was performed for each treatment alone and for the concomitant treatments using the Cytek® Northern Lights™ flow cytometer.

The results for A549 cell line showed that at most time points both TTFields and nedisertib induce G1 arrest and a decreased number of s-phase cells, and the concomitant treatment results in the highest level of arrest. According to the low levels of apoptosis measured in this nedisertib concentration, we don't see a significant change in the sub G1 phase.

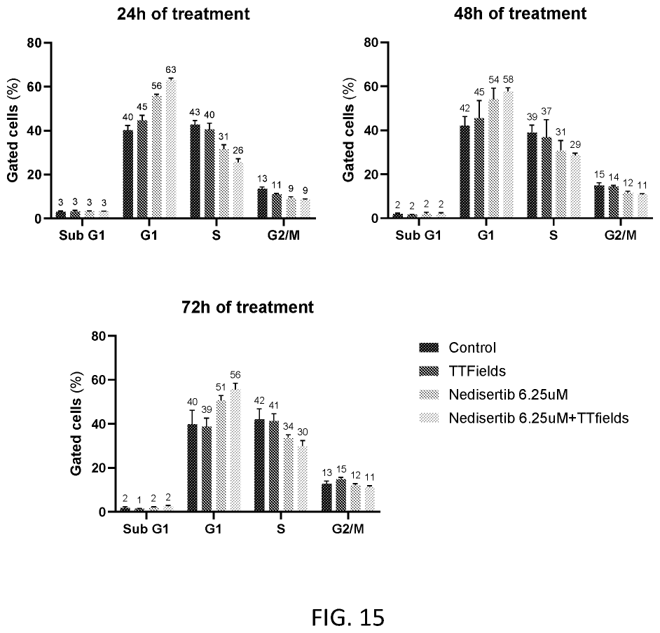

Similar results to the A549 cells were obtained in the H1299 cell line, where the concomitant treatment results in G1 arrest (FIG. 15). In these experiments 15,000 of H1299 cells/dish were seeded in inovitro dishes and incubated overnight to allow cell attachment. The cells were then treated with TTFields (0.97 V/cm RMS. 150 kHz) for different times, alone or with the addition of 6.25 μM of nedisertib. Cell cycle distribution was performed for each treatment alone and for the concomitant treatments using the Cytek® Northern Lights™ flow cytometer.

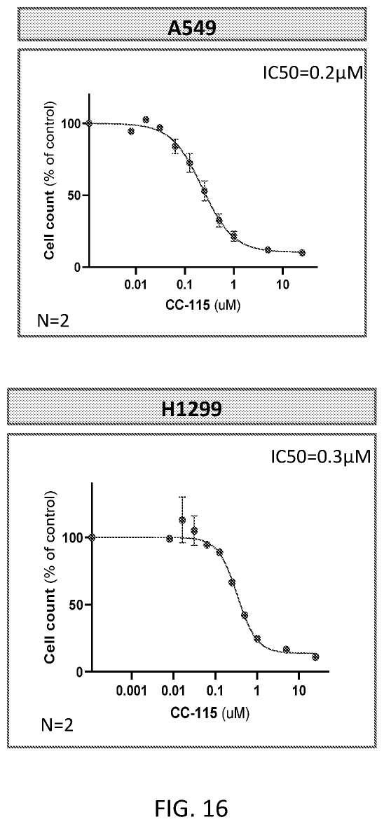

The work also involved the titration of CC-115 (FIG. 16). For these experiments 15,000 of A549 or H1299 cells/dish were seeded in 12-well plates and incubated overnight to allow cell attachment. The cells were then treated with various concentrations of CC-115 for 72 hr. The remaining cells were counted by flow cytometer (Guava easy Cyte HT). The IC50 for CC-115 in both cell lines is much lower than that of nedisertib and was similar to previously reported results. Here again, the IC50 for H1299 was higher than that of A549.

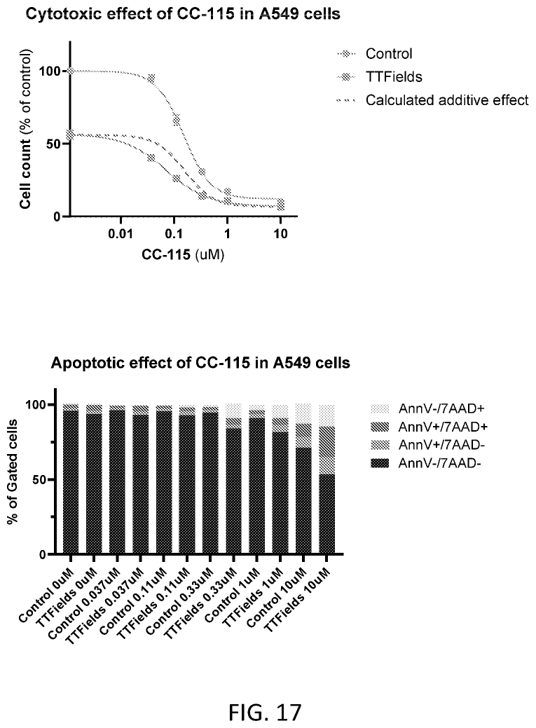

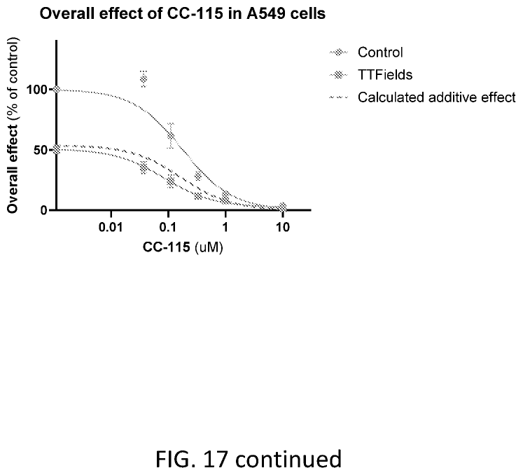

Similarly to nedisetib, in the A549 cell line we can see that TTFields synergistically enhanced the cytotoxic and overall effect of CC-115 (FIG. 17). The synergistic effect in cytotoxicity is more profound compared to nedisertib, which is expected due to the dual inhibitory effect of CC-115. In these experiments 15,000 of A549 cells/dish were seeded in inovitro dishes and incubated overnight to allow cell attachment. The cells were then treated with TTFields (0.97 V/cm RMS, 150 kHz, 72 h), alone or with the addition of different concentrations of CC-115. Efficacy was measured by analyzing cell count by flow cytometer (Guava easy Cyte HT), colony formation, and apoptosis induction by annexin V/7AAD staining. The overall effect was calculated by multiplying cell count with colony formation.

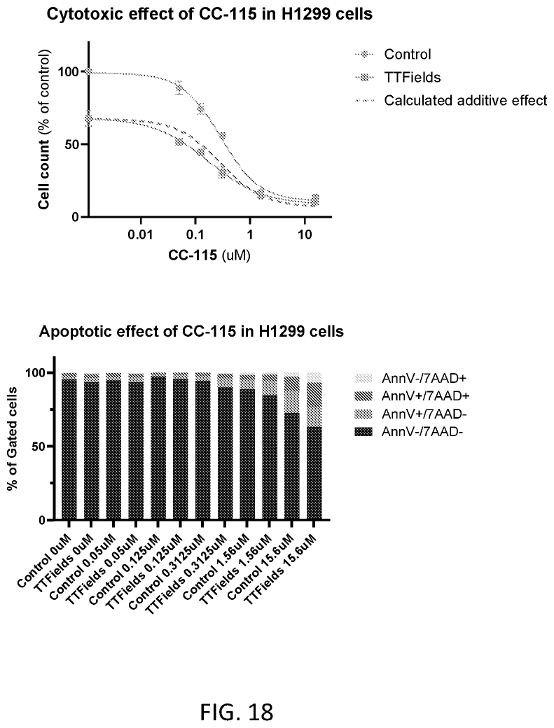

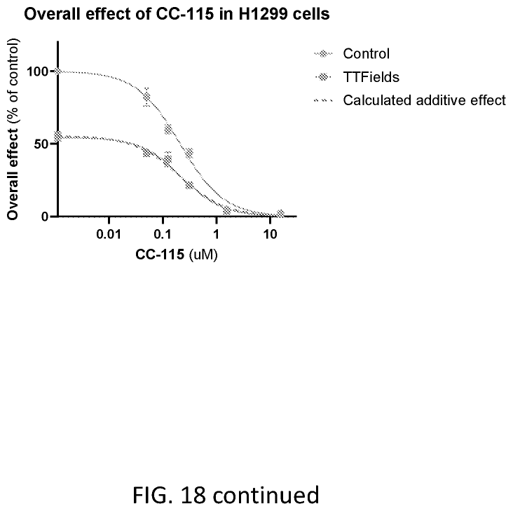

As for H1299, we can see a synergistical effect in cytotoxicity for the concomitant treatment of TTFields and CC-115 and an additive overall effect (FIG. 18). In these experiments 15,000 of H1299 cells/dish were seeded in inovitro dishes and incubated overnight to allow cell attachment. The cells were then treated with TTFields (0.97 V/cm RMS, 150 kHz, 72 h), alone or with the addition of different concentrations of CC-115. Efficacy was measured by analyzing cell count by flow cytometer (Guava easy Cyte HT), colony formation, and apoptosis induction by annexin V/7AAD staining. The overall effect was calculated by multiplying cell count with colony formation.

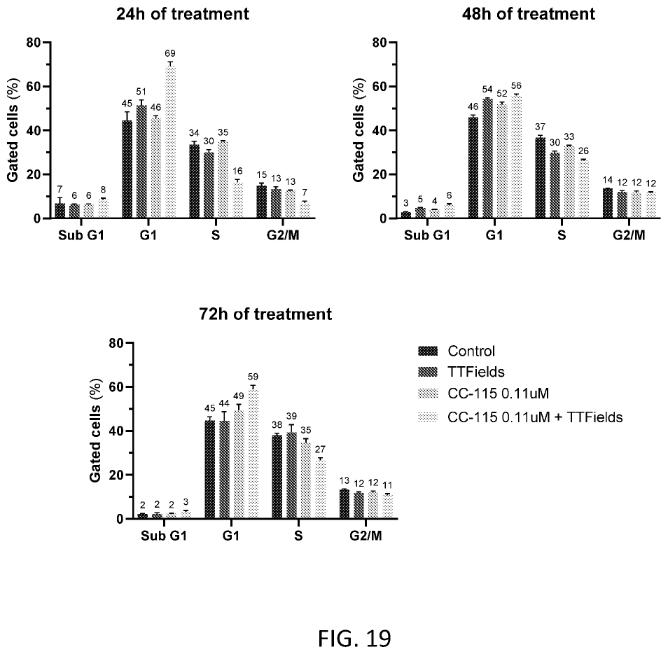

The concomitant treatment of CC-115 and TTFields that affect the cell cycle of the cells has been investigated (FIG. 19). In these experiments, 15,000 of A549 cells/dish were seeded in inovitro dishes and incubated overnight to allow cell attachment. The cells were then treated with TTFields (0.97 V/cm RMS, 150 kHz) for different times, alone or with the addition of 0.11 μM of CC-115. Cell cycle distribution was performed for each treatment alone and for the concomitant treatments using the Cytek® Northern Lights™ flow cytometer. The results for A549 cell line showed that the concomitant treatment induced G1 arrest and a decreased number of s-phase cells. Also here, according to the low levels of apoptosis measured in this CC-115concentration, we don't see a significant change in the sub G1 phase.

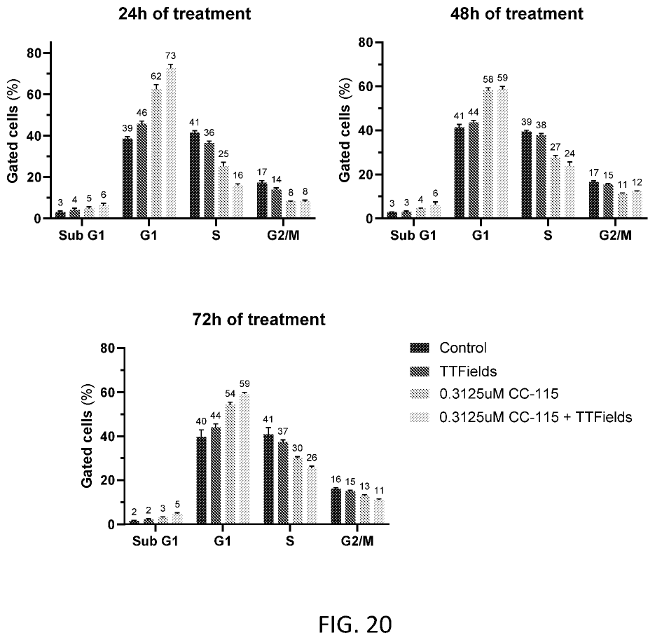

Similar results were obtained in the H1299 cell line, where the concomitant treatment results in G1 arrest (FIG. 20). In these experiments 15,000 of H1299 cells/dish were seeded in inovitro dishes and incubated overnight to allow cell attachment. The cells were then treated with TTFields (0.97 V/cm RMS, 150 kHz) for different times, alone or with the addition of 0.3125 μM of CC-115. Cell cycle distribution was performed for each treatment alone and for the concomitant treatments using the Cytek® Northern Lights™ flow cytometer.

Co-application of TTFields and Nedisertib or CC-115 showed a synergistic interaction in A549 cells. In H1299 cells, the concomitant treatment of TTFields and Nedisertib showed additive interaction and the concomitant treatment of TTFields and CC-115 showed synergistic interaction in cytotoxicity and an additive overall effect.

### ILLUSTRATIVE EMBODIMENTS

One example of the many embodiments described herein is a method of treating a subject having cancer comprising applying an alternating electric field to a target site of the subject for a period of time, wherein the target site comprises one or more cancer cells, and administering a therapeutically effective amount of a DNA-dependent PK inhibitor to the subject.

In one example of the many embodiments described herein the cancer is ovarian cancer, hepatobiliary cancer, prostate cancer, pancreatic cancer, head and neck cancers, glioblastoma, gliosarcoma, leukemia, or non-small cell lung cancer.

In one example of the many embodiments described herein the subject having cancer has undergone or is currently undergoing radiation therapy or chemotherapy.

In one example of the many embodiments described herein the one or more cancer cells at the target site have a DNA strand break, for example a single stranded DNA break or a double stranded DNA break.

In one example of the many embodiments described herein the one or more cancer cells at the target site have double stranded DNA breaks.

In one example of the many embodiments described herein at least one DNA repair mechanism in the one or more cancer cells at the target site is inhibited.

In one example of the many embodiments described herein the DNA repair mechanism inhibited is homologous recombination repair, non-homologous end joining repair, or both.

In one example of the many embodiments described herein one or more cancer cells at the target site undergo cell death.

In one example of the many embodiments described herein the therapeutically effective amount of a DNA-dependent protein kinase (PK) inhibitor is administered orally, subcutaneously or intravenously.

One example of the many embodiments described herein is a method of inducing cell death of a cancer cell comprising exposing the cancer cell to an alternating electric field for a period of time, and exposing the cancer cell to a DNA-dependent PK inhibitor.

In one example of the many embodiments described herein the cancer cell is simultaneously exposed to radiation or was previously exposed to radiation.

One example of the many embodiments described herein is a method of inhibiting DNA repair in a cancer cell exposed to or previously exposed to radiation comprising exposing the cancer cell to an alternating electric field for a period of time, and exposing the cancer cell to a DNA-dependent PK inhibitor.

In one example of the many embodiments described herein the cancer cell is in a subject.

In one example of the many embodiments described herein exposing the cancer cell to an alternating electric field comprises administering the alternating electric field to a target site of the subject, wherein the target site comprises one or more cancer cells.

In one example of the many embodiments described herein the cancer cell is from ovarian cancer, hepatobiliary cancer, prostate cancer, pancreatic cancer, head and neck cancers, glioblastoma, gliosarcoma, leukemia, or non-small cell lung cancer.

In one example of the many embodiments described herein the DNA-dependent PK inhibitor is Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026 or NU 7441 or a combination thereof.

In one example of the many embodiments described herein the methods further comprise administering to the subject a therapeutically effective amount of an ATR inhibitor, ATM inhibitor, or a PARP inhibitor.

In one example of the many embodiments described herein the ATR inhibitor is Schisandrin B, Nu6027, Dactolisib, EPT-46464, VE-821, AZ20, Berzosertib, Torin-2,Ceralasertib (AZD6738), Tetrahydropyrazolo [1,5-a]pyrazines, Azabenzimidazoles, Gartisertib, (M4344 or VX-803), Bayl895344 (Elimsuretib), CGK 733, Camonsertib, ATR-IN-4, VE-821, AZ20, ETP-46464, or ATR inhibitor 1.

In one example of the many embodiments described herein the ATM inhibitor is M4076, KU-55933, KU-60019, CP-466722, Wortmannin, Torin 2, AZD0156, or AZ31.

In one example of the many embodiments described herein the PARP inhibitor is Olaparib, niraparib, talazoparib, rucaparib, or AZD9574.

In one example of the many embodiments described herein the therapeutically effective amount of VX-984 is 50-720 mg/day.

In one example of the many embodiments described herein the cancer cells have one or more DNA strand breaks, for example single stranded DNA breaks and/or double stranded DNA breaks.

In one example of the many embodiments described herein the cancer cells have double stranded DNA breaks.

In one example of the many embodiments described herein at least one DNA repair mechanism of the cancer cells is inhibited.

In one example of the many embodiments described herein the DNA repair mechanism inhibited is homologous recombination repair, nonhomolgous end joining repair, or both.

In one example of the many embodiments described herein the cancer cells undergo cell death.

In one example of the many embodiments described herein the alternating electric field has a frequency and field strength.

In one example of the many embodiments described herein the frequency is between 100 KHz and 1 MHz.

In one example of the many embodiments described herein the frequency is between 100 and 500 KHz.

One example of the many embodiments described herein is a method of increasing the efficacy of radiation therapy in a subject comprising applying an alternating electric field to a target site of the subject for a period of time, the alternating electric field having a frequency and field strength, wherein the target site comprises a site receiving or that has received radiation therapy, and administering a therapeutically effective amount of an DNA-dependent protein kinase (PK) inhibitor to the subject.

In one example of the many embodiments described herein the target site comprises one or more cancer cells.

In one example of the many embodiments described herein the subject has cancer.

In one example of the many embodiments described herein the cancer is ovarian cancer, hepatobiliary cancer, prostate cancer, pancreatic cancer, head and neck cancers, glioblastoma, gliosarcoma, leukemia, or non-small cell lung cancer.

In one example of the many embodiments described herein the one or more cancer cells at the target site have one or more DNA strand breaks, for example single stranded DNA breaks and/or double stranded DNA breaks.

In one example of the many embodiments described herein the one or more cancer cells at the target site have double stranded DNA breaks.

In one example of the many embodiments described herein at least one DNA repair mechanism in the one or more cancer cells at the target site is inhibited.

In one example of the many embodiments described herein the DNA repair mechanism inhibited is homologous recombination repair, non-homologous end joining repair, or both.

In one example of the many embodiments described herein one or more cancer cells at the target site undergo cell death.

In one example of the many embodiments described herein the therapeutically effective amount of a DNA-dependent protein kinase (PK) inhibitor is administered orally, subcutaneously or intravenously.

In one example of the many embodiments described herein the DNA-dependent PK inhibitor is Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026 or NU 7441 or a combination thereof.

In one example of the many embodiments described herein the therapeutically effective amount of VX-984 is 50-720 mg/day.

In one example of the many embodiments described herein the cancer cells have one or more DNA strand breaks, for example single stranded DNA breaks and/or double stranded DNA breaks.

In one example of the many embodiments described herein the cancer cells have double stranded DNA breaks.

In one example of the many embodiments described herein at least one DNA repair mechanism of the cancer cells is inhibited.

In one example of the many embodiments described herein the DNA repair mechanism inhibited is homologous recombination repair, nonhomolgous end joining repair, or both.

In one example of the many embodiments described herein the cancer cells undergo cell death.

In one example of the many embodiments described herein the alternating electric field has a frequency and field strength.

In one example of the many embodiments described herein the frequency is between 100 kHz and 1 MHz.

In one example of the many embodiments described herein the frequency is between 100 and 500 KHz.

One example of the many embodiments described herein is a DNA-dependent PK inhibitor for use in a method of treating a subject in need thereof, the method comprising applying alternating electric fields, to a target site of the subject in need thereof; and administering a DNA-dependent PK inhibitor to the subject in need thereof.

One example of the many embodiments described herein is a combination of alternating electric fields and DNA-dependent PK inhibitor for use in the treatment of a subject in need thereof.

One example of the many embodiments described herein is a DNA-dependent PK inhibitor for use in a method of inducing cell death a subject in need thereof, the method comprising applying alternating electric fields, to a target site of the subject in need thereof; and administering a DNA-dependent PK inhibitor to the subject in need thereof.

One example of the many embodiments described herein is a combination of alternating electric fields and DNA-dependent PK inhibitor for use in a method of inducing cell death a subject in need thereof.

One example of the many embodiments described herein is a DNA-dependent PK inhibitor for use in a method of inhibiting DNA repair in a subject in need thereof, the method comprising applying alternating electric fields, to a target site of the subject in need thereof; and administering a DNA-dependent PK inhibitor to the subject in need thereof.

One example of the many embodiments described herein is a combination of alternating electric fields and DNA-dependent PK inhibitor for use in a method of inhibiting DNA repair in a subject in need thereof.

One example of the many embodiments described herein is a DNA-dependent PK inhibitor for use in a method of increasing efficacy of radiation therapy in a subject in need thereof, the method comprising applying alternating electric fields, to a target site of the subject in need thereof; and administering a DNA-dependent PK inhibitor to the subject in need thereof.

One example of the many embodiments described herein is a combination of alternating electric fields and DNA-dependent PK inhibitor for use in a method of increasing efficacy of radiation therapy in a subject in need thereof.

In one example of the many embodiments described herein the alternating electric fields are applied at a frequency for a period of time to a target site of the subject in need thereof.

In one example of the many embodiments described herein the subject has cancer. In some aspects, the cancer can be ovarian cancer, hepatobiliary cancer, prostate cancer, pancreatic cancer, head and neck cancers, glioblastoma, gliosarcoma, leukemia, or non-small cell lung cancer.

In one example of the many embodiments described herein the target site comprises one or more cancer cells.

In one example of the many embodiments described herein the alternating electric fields are applied before, after, or simultaneously with administering the one or more DNA-dependent PK inhibitors.

In one example of the many embodiments described herein the cancer is ovarian cancer, hepatobiliary cancer, prostate cancer, pancreatic cancer, head and neck cancers, glioblastoma, gliosarcoma, leukemia, or non-small cell lung cancer.

In one example of the many embodiments described herein the subject having cancer has undergone or is currently undergoing radiation therapy or chemotherapy.

In one example of the many embodiments described herein the one or more cancer cells at the target site have one or more DNA strand breaks, for example single stranded DNA breaks and/or double stranded DNA breaks.

In one example of the many embodiments described herein the one or more cancer cells at the target site have double stranded DNA breaks.

In one example of the many embodiments described herein at least one DNA repair mechanism in the one or more cancer cells at the target site is inhibited.

In one example of the many embodiments described herein the DNA repair mechanism inhibited is homologous recombination repair, non-homologous end joining repair, or both.

In one example of the many embodiments described herein one or more cancer cells at the target site undergo cell death.

In one example of the many embodiments described herein the therapeutically effective amount of a DNA-dependent protein kinase (PK) inhibitor is administered orally, subcutaneously or intravenously.

In one example of the many embodiments described herein the alternating electric field has a frequency and field strength.

In one example of the many embodiments described herein the frequency is between 100 KHz and 1 MHz.

In one example of the many embodiments described herein the frequency is between 100 and 500 KHz.

One example of the many embodiments described herein is a kit for increasing the efficacy of radiation therapy in a subject, the kit comprising: one or more of DNA-dependent protein kinase PK inhibitors; and one or more materials for delivering alternating electric fields to a target site of a subject for a period of time, the alternating electric field having a frequency and field strength, wherein the target site comprises a site receiving or that has received radiation therapy.

One further example of the many embodiments described herein is a kit for inducing cell death of a cancer cell, wherein the cancer cell is in a target site of a subject, the kit comprising: one or more of DNA-dependent protein kinase PK inhibitors; and one or more materials for delivering alternating electric fields to expose the cancer cell in the target site of the subject to the alternating electric fields for a period of time.

One still further example of the many embodiments described herein is a kit for inhibiting DNA repair in a cancer cell exposed to or previously exposed to radiation, wherein the cancer cell is in a target site of a subject, the kit comprising: one or more of DNA-dependent protein kinase PK inhibitors; and one or more materials for delivering alternating electric fields to expose the cancer cell in the target site of the subject to the alternating electric fields for a period of time.

Those skilled in the art will recognize, or be able to ascertain using no more than routine experimentation, many equivalents to the specific embodiments of the method and compositions described herein. Such equivalents are intended to be encompassed by the following claims.

1. A method of treating a subject having cancer comprising:
a) applying an alternating electric field to a target site of the subject for a period of time, wherein the target site comprises one or more cancer cells, and
b) administering a therapeutically effective amount of a DNA-dependent protein kinase (PK) inhibitor to the subject.

2. The method of claim 1, wherein the cancer is ovarian cancer, hepatobiliary cancer, prostate cancer, pancreatic cancer, head and neck cancers, glioblastoma, gliosarcoma, leukemia, or non-small cell lung cancer.

3. The method of claim 1, wherein the subject having cancer has undergone or is currently undergoing radiation therapy or chemotherapy.

4. The method of claim 1, wherein the one or more cancer cells at the target site have double stranded DNA breaks.

5. The method of claim 1, wherein at least one DNA repair mechanism in the one or more cancer cells at the target site is inhibited.

6. The method of claim 5, wherein the DNA repair mechanism inhibited is homologous recombination repair, non-homologous end joining repair, or both.

7. The method of claim 1, wherein one or more cancer cells at the target site undergo cell death.

8. The method of claim 1, wherein the therapeutically effective amount of a DNA-dependent protein kinase (PK) inhibitor is administered orally, subcutaneously or intravenously.

9. A method of inducing cell death of a cancer cell comprising:
a) exposing the cancer cell to an alternating electric field for a period of time, and
b) exposing the cancer cell to a DNA-dependent PK inhibitor.

10. The method of claim 9, wherein the cancer cell is simultaneously exposed to radiation or was previously exposed to radiation.

11. A method of inhibiting DNA repair in a cancer cell exposed to or previously exposed to radiation comprising:
c) exposing the cancer cell to an alternating electric field for a period of time, and
d) exposing the cancer cell to a DNA-dependent PK inhibitor.

12. The method of claim 9, wherein the cancer cell is in a subject.

13. The method of claim 12, wherein exposing the cancer cell to an alternating electric field comprises administering the alternating electric field to a target site of the subject, wherein the target site comprises one or more cancer cells.

14. The method of claim 9, wherein the cancer cell is from ovarian cancer, hepatobiliary cancer, prostate cancer, pancreatic cancer, head and neck cancers, glioblastoma, gliosarcoma, leukemia, or non-small cell lung cancer.

15. The method of claim 9, wherein the DNA-dependent PK inhibitor is Nedisertib, VX-984, CC-115, CC-122, AZD7648, matinib, vemurafenib, gefitinib, Peposertib (M3814), MSC2490484A, LY294002, JU-57788, CC-115, BAY-8400, SF2523, LTURM34, Compound 401, AMA-37, IC 86621, DNA-PK-IN-1/2/3/4/5/6/7/8/9, NU7026 or NU 7441 or a combination thereof.

16. The method of claim 15, wherein the therapeutically effective amount of VX-984 is 50-720 mg/day.

17. The method of claim 9, wherein at least one DNA repair mechanism of the cancer cells is inhibited.

18. The method of claim 9, wherein the cancer cells undergo cell death.

19. The method of claim 1, wherein the alternating electric field has a frequency and field strength, wherein the frequency is between 100 KHz and 1 MHz.
